(19) United States
(12) Patent Application Publication
(10) Pub. No.: US 2004/0010126 A1
(43) Pub. Date: Jan. 15, 2004

(54) Protein mapping

(75) Inventors: David M. Lubman, Ann Arbor, MI (US); Timothy J. Barder, Glen Ellyn, IL (US); Daniel B. Wall, Shrewsbury, MA (US); Stephen J. Parus, Ann Arbor, MI (US); Maureen T. Kachman, Ann Arbor, MI (US); Bathsheba E. Chong, Saint Paul, MN (US); Samir M. Hanash, Ann Arbor, MI (US); David E. Misek, Ann Arbor, MI (US); Robert Hinderer, Flint, MI (US)

Correspondence Address: MEDLEN & CARROLL, LLP, 101 HOWARD STREET, SUITE 350, SAN FRANCISCO, CA, 94105, US

(73) Assignee: The Regents of the University of Michigan, Ann Arbor, MI

(21) Appl. No.: 09/968,930

(22) Filed: Oct. 1, 2001

Related U.S. Application Data
(63) Continuation of application No. 09/778,547, filed on Feb. 7, 2001, now abandoned.
(60) Provisional application No. 60/180,911, filed on Feb. 8, 2000.

(51) Int. Cl. C07K 1/20

(52) U.S. Cl. 530/417

(57) ABSTRACT

The present invention relates to multiphase protein separation methods capable of resolving large numbers of cellular proteins. The methods of the present invention provide protein profile maps for imaging and comparing protein expression patterns. The present invention provides alternatives to traditional 2-D gel separation methods for the screening of protein profiles.

This application claims priority benefit of U.S. Provisional Appln. Ser. No. 60/180,911, filed Feb. 08, 2000, herein incorporated by reference in its entirety.

This invention was made with government support under Grant Nos. 2-R01GM49500-5 and U19CA84953 awarded by the National Institutes of Health. The Government has certain rights in the invention.

### FIELD OF THE INVENTION

The present invention relates to multiphase protein separation methods capable of resolving large numbers of cellular proteins. The methods of the present invention provide protein profile maps for imaging and comparing protein expression patterns. The present invention provides alternatives to traditional 2-D gel separation methods for the screening of protein profiles.

### BACKGROUND OF THE INVENTION

As the nucleic acid sequence of a number of genomes, including the human genome, becomes available, there is an increasing need to interpret this wealth of information. While the availability of nucleic acid sequence allows for the prediction and identification of genes, it does not explain the expression patterns of the proteins produced from these genes. The genome does not describe the dynamic processes on the protein level. For example, the identity of genes and the level of gene expression does not represent the amount of active protein in a cell nor does the gene sequence describe post-translational modifications that are essential for the function and activity of proteins. Thus, in parallel with the genome projects there has begun an attempt to understand the proteome (i.e., the quantitative protein expression pattern of a genome under defined conditions) of various cells, tissues, and species. Proteome research seeks to identify targets for drug discovery and development and provide information for diagnostics (e.g., tumor markers).

In view of the need for information about protein expression, there is a demand among researchers for new methods to produce images of proteins expressed in cells (Kahn, Science 195:369 [1995]). The current method for separation of proteins from cell lysates is two-dimensional polyacrylamide gel electrophoresis (2-D PAGE) (See e.g., O'Farrell, J. Biol. Chem., 250:4007 [1975]; Neidhardt et al., Electrophoresis 10:116[1989]; Anderson et al., Electrophoresis 12:907[1991]; and Patterson, Electrophoresis 16:1104[1995]). This method is capable of resolving over a thousand proteins and providing a pattern of spots, with each spot representing an isolated protein. The spots provide a rough measure of the isoelectric point and molecular weight of the protein. The integrated optical density of the spot provides a measure of the amount of protein present. The pattern of spots observed in the 2-D PAGE image is generally reproducible and is representative of the cell type being analyzed. When analyzing some altered forms of a given cell type, observing changes in the 2D-PAGE pattern can reveal changes in protein expression.

While 2-D PAGE is currently the method of choice for analyzing whole cell protein expression, the technique has several important limitations. For example, the technique is labor-intensive and time consuming. The protein mass range can extend above 200 kDa, but the spot resolution and sensitivity decrease with decreasing protein molecular weight. This often means that low molecular weight proteins may not be observed on a 2-D PAGE image and that they are more likely to be unresolved from one another. Also, protein solubility and protein recovery are concerns with the 2-D gel method because hydrophobic proteins may not be observed with this technique.

Another limitation of 2-D PAGE is the amount of protein loaded per gel which is generally below 250 μg. The amount of protein in any given spot may therefore be too low for further analysis (See e.g., Damerval, Electrophoresis 15:1573[1994]). For Coomassie brilliant blue (CBB) stained gels the limit of detection is 100 ng per spot while for silver stained gels the limit of detection is 1-10 ng. Furthermore, proteins that have been isolated in 2-D gels are embedded inside the gel structure and are not free in solution, thus making it difficult to extract the protein for further analysis. Because of these limitations, the art is in need of protein mapping methods that are more efficient and have broader resolution capabilities than presently available technologies.

### SUMMARY OF THE INVENTION

The present invention relates to multiphase protein separation methods capable of resolving large numbers of cellular proteins. The methods of the present invention provide protein profile maps for imaging and comparing protein expression patterns. The present invention provides alternatives to traditional 2-D gel separation methods for the screening of protein profiles.

For example, the present invention provides a method for displaying proteins comprising providing: a sample comprising a plurality of proteins, a first separating apparatus, wherein the first separating apparatus is capable of (i.e., is configured for) separating proteins based on a first physical property, and a second separating apparatus, wherein the second separating apparatus is a liquid phase separating apparatus and wherein the second separating apparatus is capable of (i.e., is configured for) separating proteins based on a second physical property; treating the sample with the first separating apparatus to produce a first separated protein sample; treating the first separated protein sample with the second separating apparatus to produce a second separated protein sample; and displaying at least a portion of the second separated protein sample under conditions such that the first and the second physical properties of at least a portion of the plurality of proteins are revealed. In some preferred embodiments, the first and the second physical properties include, but are not limited to, charge, hydrophobicity, and molecular weight. In some embodiments, the displaying results in a two-dimensional display while in other embodiments the display it three-dimensional (e.g., display with a third physical property) or multi-dimensional.

In some embodiments, the sample comprising a plurality of proteins further comprises a buffer, wherein said plurality of proteins are solubilized in the buffer and wherein the buffer is compatible with the first and said second separating apparatus. In some preferred embodiments, the buffer is further compatible with mass spectrometry. In some embodiments, the buffer comprises a compound of the formula n-octyl SUGARpyranoside (e.g., n-octyl C₆-C₁₂ glycopyranoside, where C₆-C₁₂ glycopyranoside is a six to twelve carbon sugar pyranoside). The sugar component is not limited to any particular sugar and includes compounds such as n-octyl β-D-glucopyranoside and n-octyl β-D-galactopyranoside.

In some preferred embodiments, the sample comprises a cell lysate (e.g., cells from animals, plants, and microorganisms; cancer cells; tissue culture cell; cells at various stages of development or differentiation; embryonic cells; tissues; etc.). However, the present invention is not limited to the use of cell lysates. For example, the sample may comprise purified and partially purified protein preparations. The present invention is also not limited in the nature of the proteins. For example, proteins may include, but are not limited to, protein fragments, polypeptides, modified proteins (e.g., lipidated, glycosylated, phosphorylated etc.), protein complexes (e.g., protein/protein, protein/nucleic acid), acid proteins, basic proteins, hydrophobic proteins, hydrophilic proteins, membrane proteins, cell surface proteins, nuclear proteins, transcription factors, structural proteins, enzymes, receptors, and the like.

In some embodiments of the present invention the first separating apparatus comprises a liquid phase separating apparatus. However, the first separating apparatus is not limited to liquid phase apparatuses. For example, the first separating apparatus may be gel-based or may be selected from methods including, but not limited to, ion exclusion, ion exchange, normal/reversed phase partition, size exclusion, ligand exchange, liquid/gel phase isoelectric focusing, and adsorption chromatography. In some preferred embodiments, the first separating apparatus comprises an isoelectric focusing apparatus. In some embodiments of the present invention, the second separating apparatus comprises reverse phase HPLC. In some preferred embodiments, the reverse phase HPLC comprises non-porous reverse phase HPLC. Certain embodiments of the present invention may utilize a second separation apparatus that is not liquid phase (e.g., gel-phase).

In some embodiments of the present invention the method further comprises the step of determining the identify of at least one protein of the second separated protein sample. Although the present invention is not limited to any particular method of determining the identify the protein, in some embodiments, the method comprises analyzing said at least one protein from the second separated protein sample with mass spectrometry.

The present invention also provides a method for characterizing proteins comprising providing: a sample comprising a plurality of proteins, a first separating apparatus, wherein the first separating apparatus is capable of (i.e., is configured for) separating proteins based on a first physical property, and a second separating apparatus, wherein the second separating apparatus is a liquid phase separating apparatus and wherein the second separating apparatus is capable of (i.e., is configured for) separating proteins based on a second physical property; treating the sample with the first separating apparatus to produce a first separated protein sample; treating the first separated protein sample with the second separating apparatus to produce a second separated protein sample; and characterizing the second separated protein sample under conditions such that the first and the second physical properties of at least a portion of the plurality of proteins are analyzed. In some embodiments, the characterizing comprises quantitating the first physical property and the second physical property for two or more proteins in the second protein sample. In other preferred embodiments, the characterizing comprises the step of analyzing at least a portion of the second separated protein sample by mass spectrometry. In yet another embodiment, the characterizing comprises the step of determining the identity of at least one protein from the second separated protein sample with mass spectrometry.

The present invention also provides a method for comparing protein expression patterns comprising providing: first and second samples comprising a plurality of proteins, a first separating apparatus, wherein the first separating apparatus is capable of (i.e., is configured for) separating proteins based on a first physical property; and a second separating apparatus, wherein the second separating apparatus is a liquid phase separating apparatus and wherein the second separating apparatus is capable of separating proteins based on a second physical property; treating the first and second samples with the first separating apparatus to produce first and second separated protein samples; treating the first and second separated protein samples with the second separating apparatus to produce third and fourth separated protein samples; and comparing the first and said second physical properties of the third separated protein sample with the first and second physical properties of the fourth separated protein sample. In some embodiments, the first and second samples are combined into a single sample prior to step b) (i.e., the samples are run together rather than in parallel or in sequence). In some embodiments, at least a portion of the proteins in the first sample comprise a first label and at least a portion of the proteins in the second sample comprises a second label. In some embodiments, the comparing comprises the step of analyzing at least a portion of the third and the fourth separated protein samples by mass spectrometry.

The present invention also provides a system comprising: a first separating apparatus, wherein the first separating apparatus is capable of (i.e., is configured for) separating proteins based on a first physical property; a first delivery apparatus capable of (i.e., configured for) receiving separated protein from the first separating apparatus; a second separating apparatus wherein the second separating apparatus is a liquid phase separating apparatus, wherein the second separating apparatus is capable of (i.e., is configured for) separating proteins based on a second physical property, and wherein the second separating apparatus is capable of (i.e., configured for) receiving proteins from the first delivery apparatus; a detection system capable of (i.e., configured for) detecting proteins produced by the second separating apparatus; a processor connected to the detection system, wherein the processor produces a data representation of the proteins produced by the second separating apparatus; and a display system capable of (i.e., configured for) displaying the data representation under conditions such that the first and second physical properties of at least a portion of the plurality of proteins are revealed. In some embodiments, the system further comprises a second delivery apparatus capable of (i.e., configured for) receiving separated protein from the second separating apparatus; and a mass spectrometry apparatus capable of (i.e., configured for) receiving protein from the second delivery apparatus.

### DESCRIPTION OF THE FIGURES

FIG. 1 shows an example 2-D protein display using Isoelectric Focusing Non-Porous Reverse Phase HPLC (IEF-NP RP HPLC) separation of human erythroleukemia cell lysate proteins in one embodiment of the present invention.

FIG. 2 shows a zoom area of a portion of the display in FIG. 1 (pI=4.2 to 7.2 and tR=6.0 to 9.0) (right panel showing banding patterns) and a corresponding example of linked HPLC data (left panel showing peaks).

FIG. 3 shows a quantification of rotofor fractions in one embodiment of the present invention.

FIG. 4 shows NP RP HPLC separation from a Rotofor fraction of HEL cell lysate in one embodiment of the present invention.

FIGS. 5A and 5B show short (5A) and long (5B) NP RP HPLC separation gradient times for a rotofor fraction of HEL cell lysate in one embodiment of the present invention.

FIG. 6 shows an example of Coomassie blue stained 2-D PAGE separation of HEL cell lysate proteins.

FIG. 7 shows a direct side-by-side comparison of IEF-NP RP HPLC (four lanes on the left) with 1-D SDS PAGE (four lane on the right) for several Rotofor fractions in certain embodiments of the present invention.

FIGS. 8A and 8B show MALDI-TOF MS tryptic peptide mass maps for α-enolase isolated by IEF-NP RP HPLC (8A) and by 2-D PAGE (8B).

### GENERAL DESCRIPTION OF THE INVENTION

The present invention relates to multiphase protein separation methods capable of resolving large numbers of cellular proteins. The methods of the present invention provide protein profile maps for imaging and comparing protein expression patterns. The present invention provides alternatives to traditional 2-D gel separation methods for the screening of protein profiles. Many limitations of traditional 2-D PAGE arise from its use of the gel as the separation media. The present invention provides alternative media for the separation that offer significant advantages over 2-D PAGE techniques. For example, in some embodiments, the present invention provides methods that use two dimensional separations, where the second dimensional separation occurs in the liquid phase, rather than 2-D PAGE techniques where the final separation occurs in gel.

In some embodiments of the present invention, proteins are separated in a first dimension using any of a large number of protein separation techniques including, but not limited to, ion exclusion, ion exchange, normal/reversed phase partition, size exclusion, ligand exchange, liquid/gel phase isoelectric focusing, and adsorption chromatography. In some embodiments of the present invention, the first dimension is a liquid phase separation method. The sample from the first separation is passed through a second dimension separation. In preferred embodiments of the present invention, the second dimension separation is conducted in liquid phase. The products from the second dimension separation are then characterized. For example, in preferred embodiments, the products of the second separation step are detected and displayed in a 2-D format based on the physical properties of the proteins that were distinguished in the first and second separation steps (e.g., under conditions such that the first and the second physical properties are revealed of at least a portion of the proteins). The products may be further analyzed, for example, by mass spectrometry to determine the mass and/or identity of the products or a subset of the products. In these embodiments, a three dimensional characterization can be applied (i.e., based on the physical properties of the first two separation steps and the mass spectrometry data). It is contemplated that other protein processing steps can be conducted at any stage of the process.

In certain embodiments of the present invention, the steps are combined in an automated system. In preferred embodiments, each of the steps is automated. For example, the present invention provides a system that includes each of the separation and detection elements in operable combination so that a protein sample is applied to the system and the user receives expression map displays or other desired data output. To achieve automation, the products of each step should be compatible with the subsequent step of steps.

In one illustrative embodiment of the present invention proteins are separated according to their pI, using isoelectric focusing (IEF) in a Rotofor and according to their hydrophobicity and molecular weight using NP RP HPLC. This combined separation method is abbreviated IEF-NP RP HPLC. When coupled with mass spectrometry (MS) this technique becomes three-dimensional and allows for the creation of a protein map that tells the pI and the molecular weight of the proteins in question. This information can be plotted in an image that also depicts protein abundance. The end result is a high-resolution image showing a complex pattern of proteins separated by pI and molecular weight and indicating relative protein abundances. This image can be used to determine how the proteins in a given cell line or tissue may change due to some disease state, pharmaceutical treatment, natural or induced differentiation, or change in environmental conditions. The image allows the observer to determine changes in pI, molecular weight, and abundance of any protein in the image. When interfaced to MS the identity of any target protein may also be obtained via enzymatic digests and peptide mass map analyses. In addition, this technique has the advantage of very high loadability (e.g., 1 gram) such that the lower abundance proteins may be detected.

In traditional 2-D PAGE separation and display techniques, the second phase separation is conducted in a gel (i.e., not a liquid phase) and the proteins are separated and detected by differences in molecular weight. In contrast, the present invention conducts the second phase separation, for example, in liquid phase. The products of the second phase separation techniques of the present invention are much more amenable to further characterization and to interpretation of data produced from the second phase. For example, in some embodiments of the present invention, the second phase is conducted using HPLC where the separated protein products are readily detected as peak fractions and interpreted and displayed in two dimensions by a computer based on the physical properties of the first and second separation steps. The products of HPLC separation, being in the liquid phase, are readily used in further detection steps (e.g., mass spectrometry). The methods of the present invention, as compared to traditional 2-D PAGE, allow more sample to be analyzed, are more efficient, facilitate automation, and allow for the analysis of proteins that are not detectable with 2-D PAGE.

For example, in one illustrative embodiment of the present invention, the protein profile of human erythroleukemia (HEL) cells has been analyzed using the methods of the present invention as well as traditional gel based methods for comparison purposes. Two-dimensional images were generated representing each of the separation methods used. Proteins were separated and then collected using both the IEF-NP RP HPLC of the present invention and 2-D PAGE methods. These proteins were then enzymatically digested and the peptide mass maps were determined by MALDI-TOF MS (if a protein cannot be unambiguously identified by this method, further analysis is made by any number of techniques including, but not limited to, LC/MS-MS, PSD-MALDI, NMR, Western blotting, Edman sequence analysis and mass spectrometry can help with further analysis of proteins [See e.g., Yates, J. Mass Spec., 33:1 (1998); Chen et al., Rap. Comm. Mass Spec., 13:1907 (1999); Neubauer and Mann, Anal. Chem. 71:235 (1999); Zugaro et al., Electrophoresis 19:867 (1998); Immler et al., Electrophoresis 19:1015 (1998); Reid et al., Electrophoresis 19:946 (1998); Rosenfeld, et al., Anal. Biochem., 203:173 (1992); Matsui et al., Electrophoresis 18:409 (1997); Patterson and Aebersold, Electrophoresis 16:1791 (1995)]).

The proteins were tentatively identified using MS-Fit to search the peptide mass maps against the Swiss and NCBInr protein databases. This work demonstrated that a large number of proteins, with a useful mass range, were separated using the methods of the present invention and that a 2-D image of these proteins was reproducibly generated for the purpose of observing distinctive patterns that are associated with a particular cell line. The methods of the present invention allowed for the detection of proteins not observed with the 2-D PAGE technique. Automation and speed of analysis are also greatly facilitated given that the proteins remain in the liquid phase throughout the separation. Thus, the methods of the present invention are shown to be an advantageous technique for the generation of images of protein expression profiles as well as for the collection of individual proteins for further analyses. These capabilities allow one to monitor changes in protein expression that are linked to differentiation pathways as well as particular conditions such as cancer (See e.g., Hanash, Advances in Electrophoresis; Chrambach, A., Editor, pp 1-44[1998]), cell aging (See e.g., Steller, Science 267:1445[1995]), the response of cells to environmental insult (See e.g., Welsh et al., Biol. Reprod., 55:141[1996]), or the response of cells to some pharmaceutical agent. Having identified significant changes in protein expression, one can then further analyze proteins of interest to determine their identity and whether they have been altered from their expected structure by sequence changes or post-translational modifications.

Definitions

To facilitate an understanding of the present invention, a number of terms and phrases are defined below:

As used herein, the term “multiphase protein separation” refers to protein separation comprising at least two separation steps. In some embodiments, multiphase protein separation refers to two or more separation steps that separate proteins based on different physical properties of the protein (e.g., a first step that separates based on protein charge and a second step that separates based on protein hydrophobicity).

As used herein, the term “protein profile maps” refers to representations of the protein content of a sample. For example, “protein profile map” includes 2-dimensional displays of total protein expressed in a given cell. In some embodiments, protein profile maps may also display subsets of total protein in a cell. Protein profile maps may be used for comparing “protein expression patterns” (e.g., the amount and identity of proteins expressed in a sample) between two or more samples. Such comparing find use, for example, in identifying proteins that are present in one sample (e.g., a cancer cell) and not in another (e.g., normal tissue), or are over- or under-expressed in one sample compared to the other.

As used herein, the term “separating apparatus capable of separating proteins based on a physical property” refers to compositions capable of separating proteins (e.g., at least one protein) from one another based on differences in a physical property between proteins present in a sample containing two or more protein species. For example, a variety of protein separation columns and composition are contemplated including, but not limited to ion exclusion, ion exchange, normal/reversed phase partition, size exclusion, ligand exchange, liquid/gel phase isoelectric focusing, and adsorption chromatography. These and other apparatuses are capable of separating proteins from one another based on their size, charge, hydrophobicity, and ligand binding affinity, among other properties. A “liquid phase” separating apparatus is a separating apparatus that utilizes protein samples contained in liquid solution, wherein proteins remain solubilized in liquid phase during separation and wherein the product (e.g., fractions) collected from the apparatus are in the liquid phase. This is in contrast to gel electrophoresis apparatuses, wherein the proteins enter into a gel phase during separation. Liquid phase proteins are much more amenable to recovery/extraction of proteins as compared to gel phase. In some embodiments, liquid phase proteins samples may be used in multi-step (e.g., multiple separation and characterization steps) processes without the need to alter the sample prior to treatment in each subsequent step (e.g., without the need for recovery/extraction and resolubilization of proteins).

As used herein, the term “displaying proteins” refers to a variety of techniques used to interpret the presence of proteins within a protein sample. Displaying includes, but is not limited to, visualizing proteins on a computer display representation, diagram, autoradiographic film, list, table, chart, etc. “Displaying proteins under conditions that first and second physical properties are revealed” refers to displaying proteins (e.g., proteins, or a subset of proteins obtained from a separating apparatus) such that at least two different physical properties of each displayed protein are revealed or detectable. For example, such displays include, but are not limited to, tables including columns describing (e.g., quantitating) the first and second physical property of each protein and two-dimensional displays where each protein is represented by an X,Y locations where the X and Y coordinates are defined by the first and second physical properties, respectively, or vice versa. Such displays also include multi-dimensional displays (e.g., three dimensional displays) that include additional physical properties.

As used herein, “characterizing protein samples under conditions such that first and second physical properties are analyzed” refers to the characterization of two or more proteins, wherein two different physical properties are assigned to each analyzed (e.g., displayed, computed, etc.) protein and wherein a result of the characterization is the categorization (i.e., grouping and/or distinguishing) of the proteins based on these two different physical properties.

As used herein, the term “comparing first and second physical properties of separated protein samples” refers to the comparison of two or more protein samples (or individual proteins) based on two different physical properties of the proteins within each protein sample. Such comparing includes grouping of proteins in the samples based on the two physical properties and comparing certain groups based on just one of the two physical properties (i.e., the grouping incorporates a comparison of the other physical property).

As used herein, the term “delivery apparatus capable of receiving a separated protein from a separating apparatus” refers to any apparatus (e.g., microtube, trough, chamber, etc.) that receives one or more fractions or protein samples from a protein separating apparatus and delivers them to another apparatus (e.g., another protein separation apparatus, a reaction chamber, a mass spectrometry apparatus, etc.).

As used herein, the term “detection system capable of detecting proteins” refers to any detection apparatus, assay, or system that detects proteins derived from a protein separating apparatus (e.g., proteins in one or fractions collected from a separating apparatus). Such detection systems may detect properties of the protein itself (e.g., UV spectroscopy) or may detect labels (e.g., fluorescent labels) or other detectable signals associated with the protein. The detection system converts the detected criteria (e.g., absorbance, fluorescence, etc.) of the protein into a signal that can be processed or stored electronically or through similar means.

As used herein, the term “buffer compatible with an apparatus” and “buffer compatible with mass spectrometry” refer to buffers that are suitable for use in such apparatuses (e.g., protein separation apparatuses) and techniques. A buffer is suitable where the reaction that occurs in the presence of the buffer produces a result consistent with the intended purpose of the apparatus or method. For example, a buffer compatible with a protein separation apparatus solubilizes the protein and allows proteins to be separated and collected from the apparatus. A buffer compatible with mass spectrometry is a buffer that solubilizes the protein or protein fragment and allows for the detection of ions following mass spectrometry. A suitable buffer does not substantially interfere with the apparatus or method so as to prevent its intended purpose and result (i.e., some interference may be allowed).

As used herein, the term “sample” is used in its broadest sense. In one sense it can refer to a cell lysate. In another sense, it is meant to include a specimen or culture obtained from any source, including biological and environmental samples. Biological samples may be obtained from animals (including humans) and encompass fluids, solids, tissues, and gases. Biological samples include blood products (e.g., plasma and serum), saliva, urine, and the like and includes substances from plants and microorganisms. Environmental samples include environmental material such as surface matter, soil, water, and industrial samples. These examples are not to be construed as limiting the sample types applicable to the present invention.

### DETAILED DESCRIPTION OF THE INVENTION

The present invention provides a novel multi-dimensional separation method that is capable of resolving large numbers of cellular proteins. The first dimension separates proteins based on a first physical property. For example, in some embodiments of the present invention proteins are separated by pI using isoelectric focusing in the first dimension (See e.g., Righetti, Laboratory Techniques in Biochemistry and Molecular Biology; Work, T. S.; Burdon, R. H., Elsevier: Amsterdam, p 10[1983]). However, the first dimension may employ any number of separation techniques including, but not limited to, ion exclusion, ion exchange, normal/reversed phase partition, size exclusion, ligand exchange, liquid/gel phase isoelectric focusing, and adsorption chromatography. In some embodiments (e.g., some automated embodiments), it is preferred that the first dimension be conducted in the liquid phase to enable products of the separation step to be fed directly into a second liquid phase separation step.

The second dimension separates proteins based on a second physical property (i.e., a different property than the first physical property) and is preferably conducted in the liquid phase (e.g., liquid-phase size exclusion). For example, in some embodiments of the present invention proteins are separated by hydrophobicity using non-porous reversed phase HPLC in the second dimension (See e.g., Liang et al., Rap. Comm. Mass Spec., 10:1219[1996]; Griffin et al., Rap. Comm. Mass Spec., 9:1546 [1995]; Opiteck et al., Anal. Biochem. 258:344[1998]; Nilsson et al., Rap. Comm. Mass Spec., 11:610[1997]; Chen et al., Rap. Comm. Mass Spec., 12:1994[1998]; Wall et al., Anal. Chem., 71:3894[1999]; Chong et al., Rap. Comm. Mass Spec., 13:1808[1999]). This method provides for exceptionally fast and reproducible high-resolution separations of proteins according to their hydrophobicity and molecular weight. The non-porous (NP) silica packing material used in these reverse phase (RP) separations eliminates problems associated with porosity and low recovery of larger proteins, as well as reducing analysis times by as much as one third. Separation efficiency remains high due to the small diameter of the spherical particles, as does the loadability of the NP RP HPLC columns. However, the second dimension may employ any number of separation techniques. For example, in one embodiment, 1-D SDS PAGE lane gel is used. Having the second dimension conducted in the liquid phase facilitates efficient analysis of the separated proteins and enables products to be fed directly into additional analysis steps (e.g., directly into mass spectrometry analysis).

In certain embodiments of the present invention, proteins obtained from the second separation step are mapped using software (available from Dr. Stephen J. Parus, University of Michigan, Department of Chemistry, 930 N. University Ave., Ann Arbor, Mich. 48109-1055) in order to create a protein pattern analogous to that of the 2-D PAGE image—although based on the two physical properties used in the two separation steps rather than by a second gel-based size separation technique. In some embodiments, RP HPLC peaks are represented by bands of different intensity in the 2-D image, according to the intensity of the peaks eluting from the HPLC. In some embodiments, peaks are collected as the eluent of the HPLC separation in the liquid phase.

In some embodiments, the proteins collected from the second dimension were identified using proteolytic enzymes, MALDI-TOF MS and MSFit database searching. In an example using human erythroleukemia cell lysate, using IEF-NP RP HPLC, approximately 700 bands were resolved in a pI range from 3.2 to 9.5 and 38 different proteins with molecular weights ranging from 12 kDa to 75 kDa were identified. In comparison to a 2-D gel separation of the same human erythroleukemia (HEL) cell line lysate, the IEF-NP RP HPLC produced improved resolution of low mass and basic proteins. In addition, the proteins remained in the liquid phase throughout the separation, thus making the entire procedure highly amenable to automation and high throughput.

Certain preferred embodiments are described in detail below. These illustrative examples are not intended to limit the scope of the invention. For example, although the examples are described using human tissues and samples, the methods and apparatuses of the present invention can be used with any desired protein samples including samples from plants and microorganisms.

I. IEF-NP RP HPLC Method

The following description provides certain preferred embodiments for conducting isoelectric separation (first dimension) and NP RP HPLC separation (second dimension) according to the methods of the present invention.

A. IEF Separation

Proteins are extracted from cells using a lysis buffer. To facilitate an efficient process, this lysis buffer should be compatible with the downstream separation and analysis steps (e.g., NP RP HPLC and MALDI-TOF-MS) to allow direct use of the products from each step into subsequent steps. Such a buffer is an important aspect of automating the process. Thus, the preferred buffer should meet two criteria: 1) it solublizes proteins and 2) it is compatible with each of the steps in the separation/analysis methods. Although the present invention provides suitable buffers for use in the particular method configurations described below, one skilled in the art can determine the suitability of a buffer for any particular configuration by solubilizing protein sample in the buffer. If the buffer solubilizes the protein, the sample is run through the particular configuration of separation and detection methods desired. A positive result is achieved if the final step of the desired configuration produces detectable information (e.g., ions are detected in a mass spectrometry analysis). Alternately, the product of each step in the method can be analyzed to determine the presence of the desired product (e.g., determining whether protein elutes from the separation steps).

After extraction in the lysis buffer, proteins are initially separated in a first dimension. The goal in this step is that the proteins are isolated in a liquid fraction that is compatible with subsequent NP RP HPLC and mass spectrometry steps. In these embodiments, n-octyl β-D-glucopyranoside (OGl, from Sigma) is used in the buffer. n-octyl β-D-glucopyranoside is one of the few detergents that is compatible with both NP RP HPLC and subsequent mass spectrometry analyses. It is contemplated that detergents of the formula n-octyl SUGARpyranoside find use in these embodiments. The lysis buffer utilized was 6M urea, 2M thiourea, 1.0% n-octyl β-D-glucopyranoside, 10 mM dithioerythritol and 2.5% (w/v) carrier ampholytes (3.5 to 10 pI)). After extraction, the supernatant protein solution is loaded to a device that can separate the proteins according to their pI by isoelectric focusing (IEF). Here the proteins are solubilized in a running buffer that again should be compatible with NP RP HPLC. A suitable running buffer is 6M urea, 2M thiourea, 0.5% n-octyl β-D-glucopyranoside, 10 mM dithioerythritol and 2.5% (wlv) carrier ampholytes (3.5 to 10 pI).

Three exemplary devices that may be used for this step are:

1) Rotofor

This device (Biorad) separates proteins in the liquid phase according to their pI (See e.g., Ayala et al., Appl. Biochem. Biotech. 69:11[1998]). This device allows for high protein loading and rapid separations that require only four to six hours to perform. Proteins are harvested into liquid fractions after a 5-hour IEF separation. These liquid fractions are ready for analysis by NP RP HPLC. This device can be loaded with up to 1 g of protein.

2) Carrier Ampholyte Based Slab Gel IEF Separation with a Whole Gel Eluter

In this case the protein solution is loaded onto a slab gel and the proteins separate in to a series of gel-wide bands containing proteins of the same pI. These proteins are then harvested using a whole gel eluter (WGE, from Biorad). Proteins are then isolated in liquid fractions that are ready for analysis by NP RP HPLC. This type of gel can be loaded with up to 20 mg of protein.

3) IPG Slab Gel IEF Separation with a Whole Gel Eluter

Here the proteins are loaded onto a immobiline pI gradient slab gel and separated into a series of gel-wide bands containing proteins of the same pI. These proteins are electro-eluted using the WGE into liquid fractions that are ready for analysis by NP RP HPLC. The IPG gel can be loaded with at least 60 mg of protein.

B) Protein Separation by NP RP HPLC

Having obtained liquid fractions containing large amounts of p1-focused proteins the second dimension separation is non-porous RP HPLC. The present invention provides the novel combination of employing non-porous RP packing materials (e.g., MICRA-Platinum ODS-1 available from Eichrom Technologies, Inc.) with another RP HPLC compatible detergent (e.g., n-octyl β-D-galactopyranoside) to facilitate the multi-phase separation of the present invention. This detergent is also compatible with mass spectrometry due to its low molecular weight. The use of these types of RP HPLC columns for protein separations as a second dimension separation after IEF in order to obtain a 2-D protein separation is a novel feature of the present invention. These columns are well suited to this task as the non-porous packing they contain provides optimal protein recovery and rapid efficient separations. It should be noted that though several detergents have been mentioned thus far for increasing protein solubility while being compatible with RP HPLC there are many other different low molecular weight non-ionic detergents that could be used for this purpose. Several important features that allow the RP HPLC to work as a second dimension are as follows: The mobile phase should contain a low level of a non-ionic low molecular weight detergent such as n-octyl β-D-glucopyranoside or n-octyl β-D-galactopyranoside as these detergents are compatible with RP HPLC and also with later mass spectrometry analyses (unlike many other detergents); the column should be held at a high temperature (around 60° C.); and the column should be packed with non-porous silica beads to eliminate problems of protein recovery associated with porous packings.

C) Protein Detection and Identification via Mass Spectrometry

In some embodiments of the present invention, the products of the second separation step are further characterized using mass spectrometry. For example, the proteins that elute from the NP RP HPLC separation are analyzed by mass spectrometry to determine their molecular weight and identity. For this purpose the proteins eluting from the separation can be analyzed simultaneously to determine molecular weight and identity. A fraction of the effluent is used to determine molecular weight by either MALDI-TOF-MS or ESI-TOF (LCT, Micromass) (See e.g., U.S. Pat. No. 6,002,127). The remainder of the eluent is used to determine the identity of the proteins via digestion of the proteins and analysis of the peptide mass map fingerprints by either MALDI-TOF-MS or ESI-TOF. The molecular weight 2-D protein map is matched to the appropriate digest fingerprint by correlating the molecular weight total ion chromatograms (TIC's) with the UV-chromatograms and by calculation of the various delay times involved. The UV-chromatograms are automatically labeled with the digest fingerprint fraction number. The resulting molecular weight and digest mass fingerprint data can then be used to search for the protein identity via web-based programs like MSFit (UCSF).

D) Automation

All of the above described steps are automated, for example, into one discrete instrument. In one illustrative embodiment, the first dimension is carried out by a Rotofor, with the harvested liquid fractions being directly applied to the second dimension non-porous RP HPLC apparatus through the appropriate tubing. The products from the second dimension separation are then scanned and the data interpreted and displayed as a 2-D representation using the appropriate computer hardware and software. Alternately, the products from the second dimension fractions are sent through the appropriate microtubing to a mass spectrometry pre-reaction chamber where the samples are treated with the appropriate enzymes to prepare them for mass spectrometry analysis. The samples are then analyzed by mass spectrometry and the resulting data is received and interpreted by a processor. The output data represents any number of desired analyses including, but not limited to, identity of the proteins, mass of the proteins, mass of peptides from protein digests, dimensional displays of the proteins based on any of the detected physical criteria (e.g., size, charge, hydrophobicity, etc.), and the like. In preferred embodiments, the proteins samples are solubilized in a buffer that is compatible with each of the separation and analysis units of the apparatus. Using the automated systems of the present invention provides a protein analysis system that is an order of magnitude less expensive than analogous automation technology for use with 2-D gels (See e.g., Figeys and Aebersold, J. Biomech. Eng. 121:7[1999]; Yates, J. Mass Spectrom., 33:1[1998); and Pinto et al., Electrophoresis 21:181[2000]).

E) Software and Data Presentation

The data generated by the above listed techniques may be presented as 2-D images much like the traditional 2-D gel image. In some embodiments, the chromatograms, TIC's or integrated and deconvoluted mass spectra are converted to ASCII format and then plotted vertically, using a 256 step gray scale, such that peaks are represented as darkened bands against a white background. The scale could also be in a color format. The image generated by this method provides information regarding the pI, hydrophobicity, molecular weight and relative abundance of the proteins separated. Thus the image represents a protein pattern that can be used to locate interesting changes in cellular protein profiles in terms of pI, hydrophobicity, molecular weight and relative abundance. Naturally the image can be adjusted to show a more detailed zoom of a particular region or the more abundant protein signals can be allowed to saturate thereby showing a clearer image of the less abundant proteins. This information can be used to assess the impact of disease state, pharmaceutical treatment, and environmental conditions. As the image is automatically digitized it may be readily stored and used to analyze the protein profile of the cells in question. Protein bands on the image can be hyper-linked to other experimental results, obtained via analysis of that band, such as peptide mass fingerprints and MSFit search results. Thus all information obtained about a given 2-D image, including detailed mass spectra, data analyses, and complementary experiments (e.g., immuno-affinity and peptide sequencing) can be accessed from the original image.

The data generated by the above-listed techniques may also be presented as a simple read-out. For example, when two or more samples are compared (See, Section X, below), the data presented may detail the difference or similarities between the samples (e.g., listing only the proteins that differ in identity or abundance between the samples). In this regard, when the differences between samples (e.g., a control sample and an experimental sample) are indicative of a given condition (e.g., cancer cell, toxin exposure, etc.), the read-out may simply indicate the presence or identity of the condition. In one embodiment, the read-out is a simple ± indication of the presence of particular proteins or expression patterns associated with a specific condition that is to be analyzed.

F) IEF-NP RP HPLC in Operation

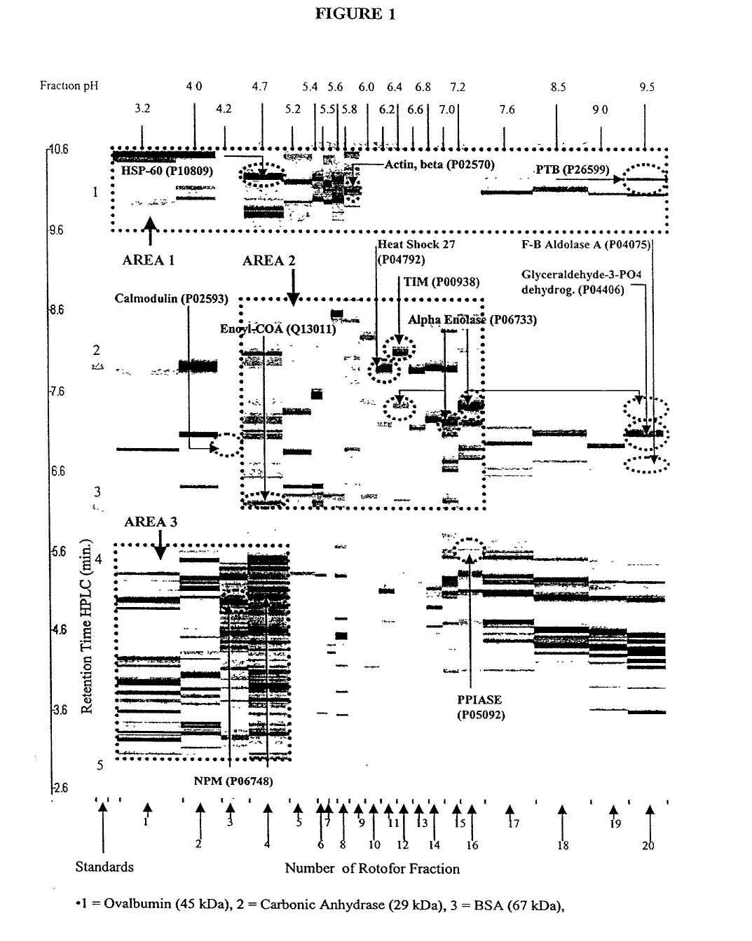

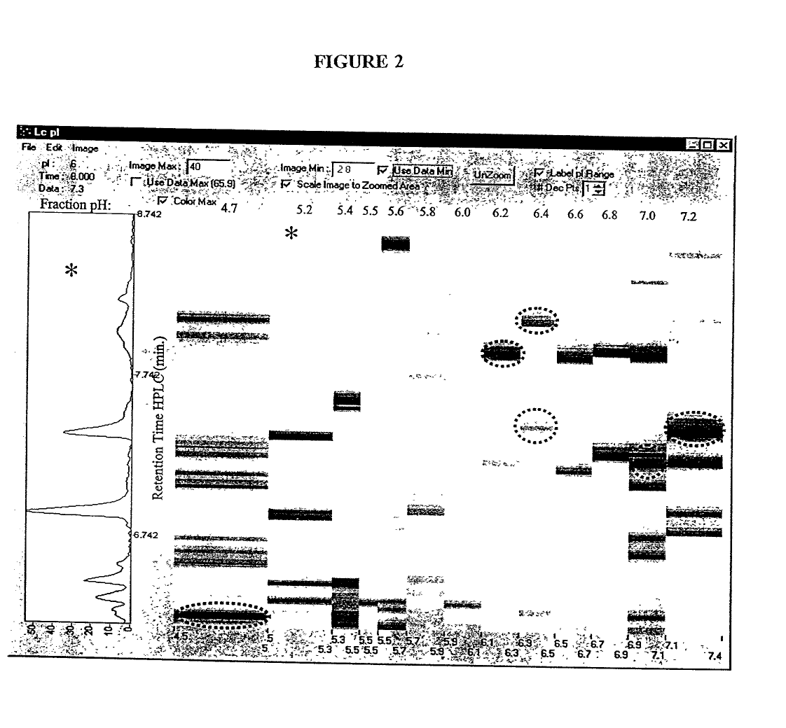

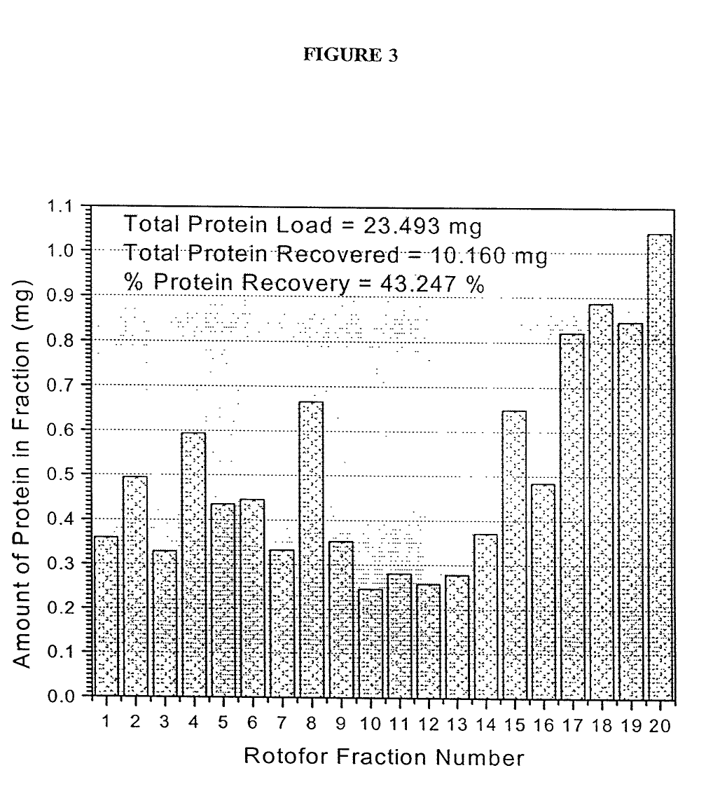

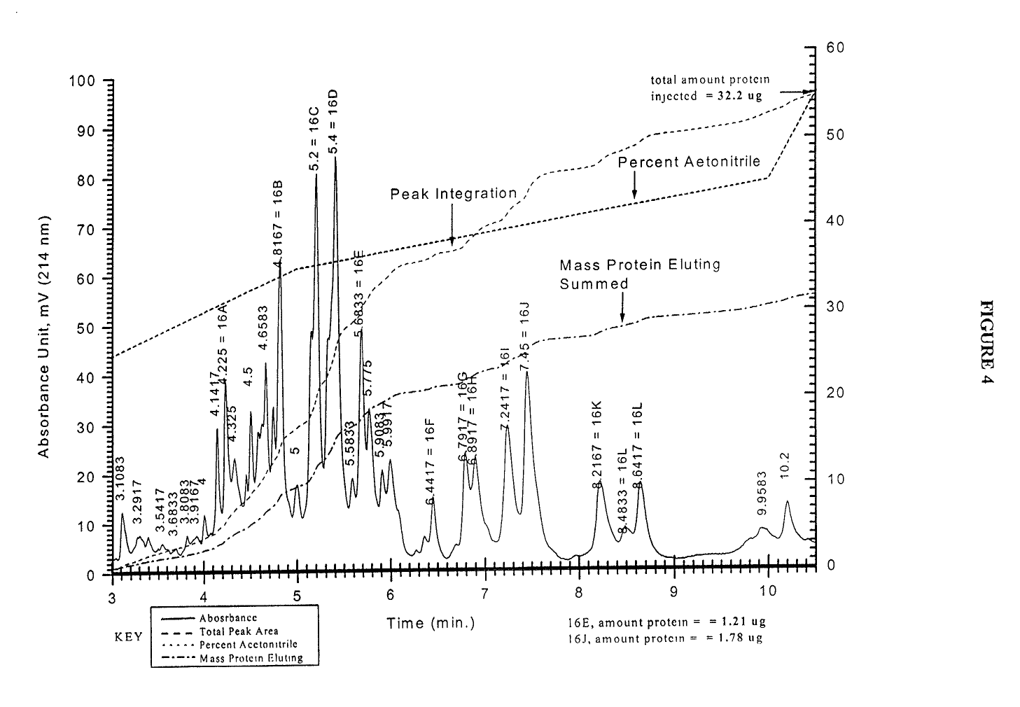

The IEF-NP RP HPLC image shown in FIG. 1 is a digital representation of a 2-dimensional separation of a whole cell protein lysate from a human erythroleukemia (HEL) cell line. This image is designed to offer the same advantages of pattern recognition and protein profiling that may be obtained using a 2-D gel. The horizontal and vertical dimensions are in terms of isoelectric point and protein hydrophobicity, respectively. The isoelectric focusing step, performed using the Rotofor, resulted in 20 protein fractions ranging in pH from 3.2 to 9.5. These fractions were then injected onto a non-porous reversed phase column for separation by HPLC and detection by UTV absorbance (214 nm). The resulting chromatograms were converted to ASCII format and then plotted vertically, using a 256 step gray scale, such that peaks are represented as darkened bands against a white background. Protein profiles may be viewed in greater detail by using the zoom feature as shown in FIG. 2 and/or by selecting a particular Rotofor fraction and observing the NP RP HPLC chromatogram as shown in the left panel of FIG. 2. The zoom and chromatogram image features provide a means to observe details in band patterns that may not be observable in the original image (See, FIG. 1). In addition, because of the limitations of the 256 step gray scale representation the band intensities in areas 1, 2 and 3 of FIG. 1 were resealed by a factor of 3 to better show the low abundance proteins. This was preferred since the presence of several high abundance protein bands may cause low intensity bands in some regions to be undetected. In FIG. 1, the total peak area for each individual chromatogram was scaled to reflect the relative amount of protein that was found in the original Rotofor fraction (See, FIG. 3). The band intensities in different chromatograms can therefore be compared directly thus providing a true image of relative protein abundance in the cell lysate. The width of the Rotofor fraction columns was adjusted to represent their estimated pH range. The molecular weight of proteins observed by IEF-NP RP HPLC ranged from 12 kDa to 75 kDa. Typical NP RP HPLC separations, as shown in FIG. 4, resulted in 35 peaks in 10.5 minutes. The total number of peaks that could be observed from all 20 fractions is estimated to be approximately 700.

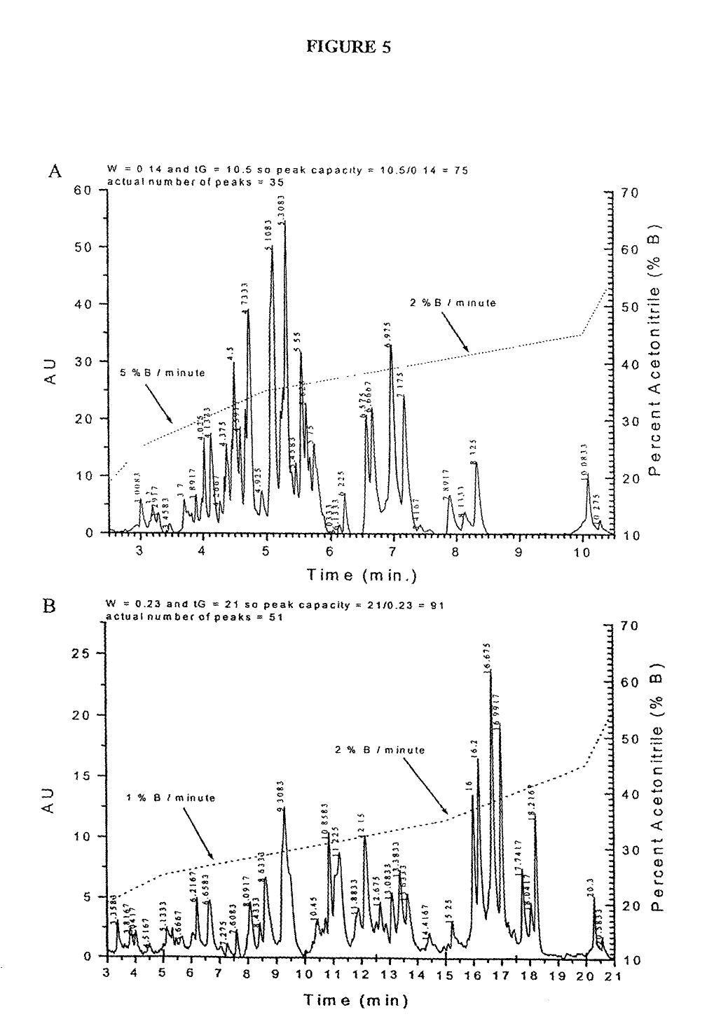

The gradient time (t_{G}) used in the above experiments is very short and a significant increase in peak capacity is expected with longer gradients. This is shown using Rotofor fraction 17 where two separations were performed with gradient times of 10.5 minutes (See, FIG. 5A) and 21 minutes (See, FIG. 5B). With t_{G}=10.5 minutes, the average peak width was 0.14 minutes and the peak capacity was therefore 75. The actual number of peaks resolved was 35. With t_{G}=21 minutes the average peak width was 0.23 minutes and the peak capacity was therefore 91. The actual number of peaks resolved was 51. Using the longer separation time with t_{G}=21 minutes the total number of peaks observed should increase from 700 to 1000. However, it should be noted that when using mass spectrometric detection, that sufficient resolution should be available to ultimately resolve the same number of peaks without using a longer gradient time.

The proteins in a representative sampling of these peaks were identified using the traditional approach of enzymatic digestion, MALDI-TOF MS peptide mass analysis and MSFit database searching. The magnification of the IEF-NP RP HPLC image enables the viewer to perceive more bands than is possible to observe from the whole image. In addition, as shown in FIG. 2, the viewer may select a particular band format chromatogram and observe the traditional peak format of the chromatogram in a window to the left of the image. This allows the observer to use the peak format chromatogram to find partially resolved peaks that may not be observable in the band format chromatogram. Five standard protein bands are shown in the left-most column where the masses range from 14.2 kDa up to 67 kDa. As RP HPLC separates proteins by hydrophobicity, these standards are not molecular weight markers as in a traditional 1-D gel. Rather, they are used to indicate the range of protein molecular weights that may be observed. Ten different proteins are labeled on the image although many more proteins were identified as shown in Table 1, below. In some embodiments of the present invention, where it is desired that certain proteins or classes of proteins are to be detected, the starting protein sample may be selectively labeled. After the proteins are passed through the separation step, detection of the proteins can be limited to those that contain the selective label.

II. Protein Separation by 2-D SDS PAGE

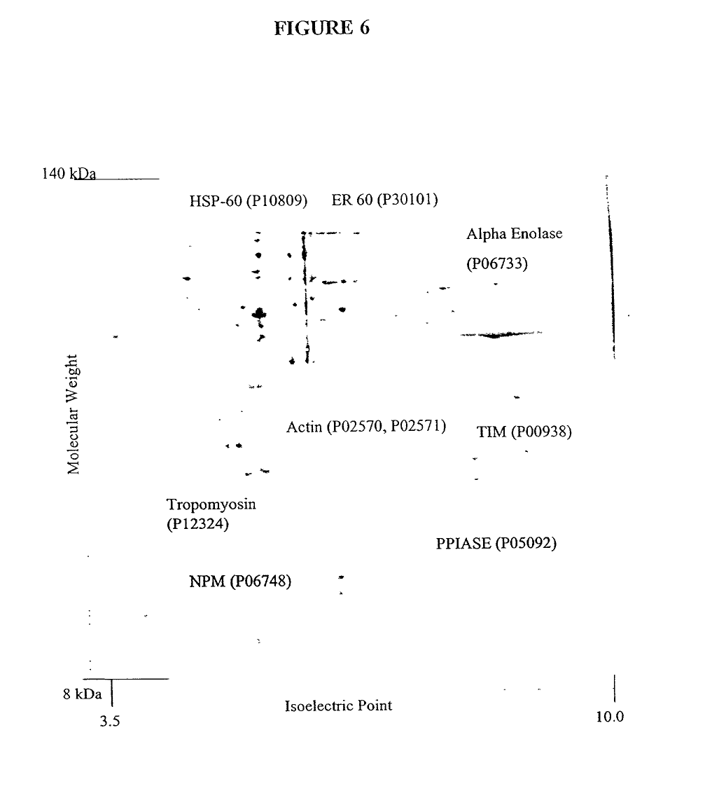

The image in FIG. 1 represents the IEF-NP RP HPLC separation of the HEL cell protein lysate and the image in FIG. 6 represents the Coomassie blue (CBB) stained 2-D SDS PAGE separation of the same HEL cell line lysate. The pI range for this gel is the same as that used for the Rotofor separation and the molecular weight range is from 8 kDa to 140 kDa. As with the IEF-NP RP HPLC separation a representative sampling of the isolated proteins was identified using enzymatic digestion, MALDI-TOF MS and MSFit methods (See e.g., Rosenfeld et al., Anal. Biochem. 203:173[1992]). For the target protein mass range of this study (10 kDa -70 kDa) approximately 188 protein spots are observed on the CBB stained gel, 355 from the CBB stained polyvinylidene difluoride (PVDF) blot, and 652 from the silver stained gel as estimated using Biolmage 2D Analyzer Version 6.1 software (Genomic Solutions). The total spot capacity for the 2-D gel separation is estimated to be 2100. The proteins identified from the gel are labeled on the image and also shown in Table 2, below. An image of another 2-D gel separation of HEL cell proteins can be observed via the Swiss-2DPAGE database (See e.g., http://www.expasy.ch; Sanchez et al., Electrophoresis 16:1131[1995]). In addition, it is possible to view the latest protein list for the HEL cell in which 19 protein entries are shown (See e.g., http://www.expasy.chlcgi-bin/get-ch2d-table.pl).

TABLE 1
Thirty Eight Proteins Identified From HEL Ccli IEF-NP RP HPLC Separation

| Rotofor | - | Retention | | MWt/pl: database | Swiss. NCBlar | |
|---|---|---|---|---|---|---|
| Fraction # | pH | Time (min.) | Enzyme * | calculated | Accession # | Protein Name |
| 3 | 4.20 | 5.34 | trypsin | 32575.2/4.64 | P06748 | NPM |
| 3 | 4.20 | 6.20 | trypsin | 11665.0/4.42 | P05387 | 60S RIBOSOMAL PROTEIN P2 |
| 3 | 4.20 | 6.91 | trypsin | 16637.7/4.09 | P02593 | CALMODULIN |
| 3 | 4.20 | 10.15 | trypsin | 41737.0/5.29 | P02570 | BETA-ACTIN & GAMMA ACTIN |
| 3 | 4.20 | 10.25 | trypsin | 61055.0/5.70 | P10809 | HSP-60 |
| 4 | 4.70 | 5.31 | trypsin | 32575.2/4.64 | P06748 | NPM |
| 4 | 4.70 | 6.24 | trypsin | 35994.6/6.61 | Q13011 | ENOYL-COA HYDRATASE |
| 4 | 4.70 | 7.07 | trypsin | 57914.2/7.95 | P14786 | PYRUVATE KINASE, M2 |
| 4 | 4.70 | 10.21 | trypsin | 61055.0/5.70 | P10809 | HSP-60 |
| S | 5.40 | 4.93 | trypsin | 22988.1/5.10 | P52566 | RHO GDI 2 |
| 5 | 5.40 | 10.15 | trypsin | 70898.4/5.38 | P11142 | HEAT SHOCK COGNATE 71 KD PROTEIN |
| 8 | 5.60 | 4.99 | trypsin | 22988.1/5.10 | P52566 | RHO GDP-DISSOCIATION INHIBITOR 2 |
| 8 | 5.60 | 7.94 | trypsin | 69224.5/5.49 | P23588 | EIF-4B |
| 8 | 5.60 | 10.35 | trypsin | 49831.3/4.79 | P05217 | TUBULIN BETA-2 CHAIN |
| 9 | 5.80 | 6.90 | trypsin | 56782.7/5.99 | P30101 | ERP60 |
| 9 | 5.80 | 8.05 | trypsin | 17148.8/5.83 | P15531 | METASTASIS INHIBITION FACTOR NM23 |
| 9 | 5.60 | 8.50 | trypsin | 26669.6/6.45 | P00938 | TRIOSEPHOSPHATE ISOMERASE (TIM) |
| 9 | 5.80 | 10.15 | trypsin | 41737.0/5.29 | P02570 | BETA-ACTIN & GAMMA ACTIN |
| 11 | 6.20 | 5.62 | trypsin | 36926.7/6.37 | 5542020 | (L32610) ribonucleoprotein |
| 11 | 6.20 | 7.65 | trypsin | 33777.2/6.26 | 4885153 | (X59656) CRKL |
| 11 | 6.20 | 7.91 | trypsin | 22327.3/7.83 | P04792 | HEAT SHOCK 27 |
| 11 | 6.20 | 8.80 | trypsin | 74674.0/8.51 | Q92935 | EXOSTOSIN-L |
| 11 | 6.20 | 9.22 | trypsin | 37374.9/5.85 | P19883 | FOLLISTATIN 1 AND 2 PRECURSOR |
| 11 | 6 20 | 10.40 | trypsin | 47033.1/5.30 | 5032183 | cargo selection protein TIP47 |
| 12 | 6.40 | 5.08 | trypsin | 13802.0/6.43 | P49773 | HINT |
| 12 | 6.40 | 5.90 | trypsin | 70021.3/5.56 | P54652 | HEAT SHOCK 70 KD PROTEIN 2 |
| 12 | 6.40 | 7.48 | trypsin | 47169.2/7.01 | P06733 | ALPHA ENOLASE |
| 12 | 6.40 | 8.12 | trypsin | 16669.6/6.45 | P00938 | TRIOSEPHOSPHATE ISOMERASE (TIM) |
| 13 | 6.60 | 4.88 | trypsin | 48058.0/5.34 | P05783 | KERATIN, TYPE 1 CYTOSKELETAL 18 |
| 13 | 6.60 | 8.28 | trypsin | 62639.6/6.40 | P31948 | TRANSFORMATION-SENSIT1VE PROTEIN |
| 13 | 6.60 | 8.65 | trypsin | 34902.4/7.42 | 4505059 | carcinoma-associated antigen GA 733-2 |
| 15 | 7.00 | 4.70 | trypsin | 37429.9/8.97 | P22626 | NUCLEAR RIBONUCLEOPROTEINS A2/B1 |
| 15 | 7.00 | 8.70 | trypsin | 22391.6/8.41 | P37802 | SM22-ALPHA HOMOLOG |
| 15 | 7.00 | 7.25 | trypsin | 47169.2/7.01 | P06733 | ALPHA ENOLASE |
| 16 | 7.20 | 5.68 | trypsin, Glu-C (E) | 18012.6/7.68 | P05092 | PPIASE |
| 16 | 7.20 | 6.89 | trypsin | 35940.7/7.18 | P01861 | IG GAMMA-4 CHAIN C REGION |
| 16 | 7.20 | 7.24 | trypsin | 36053.4/8.57 | P04406 | GLYCERALDEHYDE 3-PHOSPHATE |
| 16 | 7.20 | 7.45 | trypsin, Glu-C (E) | 47169.2/7.01 | P06733 | ALPHA ENOLASE |
| 16 | 7.20 | 8.64 | trypsin, Glu-C (E) | 22391.6/8.41 | P37802 | SM22-ALPHA HOMOLOG |
| 19 | 9.00 | 4.88 | trypsin | 38846.0/9.26 | P09651 | NUCLEAR RYBONUCLEOPROTEIN A1 |
| 19 | 9.00 | 5.13 | trypsin | 37429.9/8.97 | P22626 | NUCLEAR RIBONUCLEOPROTEINS A2/B1 |
| 19 | 9.00 | 5.85 | trypsin | 46987.1/7.58 | P13929 | BETA ENOLASE |
| 19 | 9.00 | 7.47 | trypsin | 36053.4/8.57 | P04406 | GLYCERALDEHYDE 3-PHOSPHATE |
| 19 | 9.00 | 8.70 | trypsin | 38604.2/7.58 | P07355 | ANNEXIN 11 |
| 19 | 9.00 | 9.07 | trypsin | 22391.6/8.41 | P37802 | SM22-ALPHA HOMOLOG |
| 19 | 9.00 | 10.53 | trypsin | 57221.6/9.22 | P26599 | PTB, NUCLEAR RIBONUCLEOPROTEIN 1 |
| 20 | 9.50 | 4.46 | trypsin, Glu-C (E) | 38846.0/9.26 | P09651 | NUCLEAR RIBONUCLEOPROTEIN A1 |
| 20 | 9.50 | 4.67 | trypsin, Glu-C (E) | 37429.9/8.97 | P22626 | NUCLEAR RIBONUCLEOPROTEINS A2/B1 |
| 20 | 9.50 | 6.72 | trypsin, Glu-C (E) | 39420.2/8.30 | P04075 | FRUCTOSE-BISPHOSPHATE ALDOLASE A |
| 20 | 9.50 | 7.06 | trypsin | 36053.4/8.57 | P04406 | GLYCERALDEHYDE 3-PHOSPHATE |
| 20 | 9.50 | 7.39 | trypsin, Glu-C (E) | 47169.2/7.01 | P06733 | ALPHA ENOLASE |
| 20 | 9.50 | 8.52 | trypsin, Glu-C (E) | 22391.6/8.41 | P37802 | SM22-ALPHA HOMOLOG |
| 20 | 9.50 | 10.16 | trypsin | 44728.1/8.30 | P00558 | PHOSPHOGLYCERATE KINASE 1 |
| 20 | 9.50 | 10.35 | trypsin | 57221.6/9.22 | P26599 | PTB, NUCLEAR RIBONUCLEOPROTEIN 1 |

Note that all proteins labelled only with trypsin were not digested with Glu-C (E)

TABLE 2
Nine Proteins Identified From HEL Cell CBB 2-D Gel

| Gel Spot I.D. | | MWt/pI: database | SwissProt | |
|---|---|---|---|---|
| Number | Enzyme | calculated | Accession # | Protein Name |
| g1 | trypsin | 18012.6/7.68 | P05092 | PPIASE |
| g2 | trypsin | 26669.6/6.45 | P00938 | TRIOSEPHOSPHATE ISOMERASE (TIM) |
| g3 | trypsin | 26669.6/6.45 | P00938 | TRIOSEPHOSPHATE ISOMERASE (TIM) |
| g8 | trypsin | 29032.8/4.75 | P12324 | TROPOMYOSIN, CYTOSKELETAL TYPE (TM30-NM) |
| g10 | trypsin | 32575.2/4.64 | P06748 | NPM |
| g11 | trypsin | 41737.0/5.29 | P02570 | BETA-ACTIN |
| g12 | trypsin | 61055.0/5.70 | P10809 | HSP-60 |
| g13 | trypsin | 56782.7/5.99 | P30101 | ERP60 |
| g14 | trypsin | 47169.2/7.01 | P06733 | ALPHA ENOLASE |

III. IEF-NP RP HPLC versus 2-D SDS PAGE: Protein Loading and Quantification

Each separation method relies upon orthogonal mechanisms of separation generating a large number of isolated proteins. Protein profiles may be compared in terms of their pattern as well as the relative amounts of isolated proteins. It is shown, however, that the loadability of the liquid phase methods of the present invention greatly surpasses that of the gel phase.

The limit of detection for the gel method when stained with the silver stain is approximately 1 to 10 ng. The Coomassie blue stain can detect 100 ng of protein and the amount of protein in the spot can be quantified over 2.5 orders of magnitude. For the NP RP HPLC of standard proteins used in certain embodiments of the methods of the present invention, the limit of detection for the UV detector was 10 ng. The protein in the peak can be quantified from 10 ng up to 20 μg providing 3.1 orders of magnitude. Quantification of an HPLC peak involves integrating the peak to find the area. For the gel, the spots must first be digitized and then this image must be analyzed to determine the integrated optical density of each spot of interest. The sensitivity of the UV detector in embodiments of the present invention utilizing HPLC is competitive with the silver stain and quantification is much simpler. The limits of detection for both the silver stained gel and the HPLC UV peak detection are mass dependent. For the gel, resolution and sensitivity are proportional to the molecular weight of the protein. For IEF-NP RP HPLC, the resolution and sensitivity are inversely proportional to the molecular weight of the protein. The gel appears to provide improved results for both acidic proteins and proteins above 50 kDa whereas IEF-NP RP HPLC performs better with proteins in the basic region and proteins that are below 50 kDa (See e.g., FIG. 1 and FIG. 6). These results show the complementary nature of these two techniques where the gel and IEF-NP RP HPLC each provide important information of protein content.

In one experiment using the methods of the present invention, 23.5 mg of protein was loaded into the Rotofor, and after a five-hour IEF separation period fractions ranging from 2 to 4 mL were collected into polypropylene microtubes. The amount of protein in the individual fractions ranged from 0.25 mg to 1.05 mg. Summing the amounts of protein in each fraction led to the determination that a total of 10.2 mg of protein was recovered from the Rotofor. This amount can be increased by increasing the amount of non-ionic detergent in the Rotofor buffer above the current 0.1% level as well as by the addition of thiourea. In contrast, the amount of protein loaded on the 2-D gel in FIG. 6 is 200 μg. The amount of protein that actually makes it through the gel and focuses to a spot has not been quantified, relative to the amount of protein that is actually loaded on the gel, though it is known that many hydrophobic proteins are lost during the separation (Herbert, Electrophoresis 20:660[1999]). The amount of protein that may theoretically be loaded on a gel ranges from 5 μg up to 250 μg whereas for IEF-NP RP HPLC the initial loading of protein may be as high as 1 gram. The amount of protein actually used to produce the separation shown in FIG. 1 is only a fraction of the amount initially loaded into the Rotofor. The image in FIG. 1 actually represents the separation of a total of 1 to 2 mg of protein though 10.2 mg of protein was recovered from the Rotofor. The loading of the HPLC column being used currently could be increased though the peak capacity may suffer. Alternatively a larger column could be used in series with the smaller column to allow for higher loadability with no loss of separation efficiency (See e.g., Wall et al., Anal. Chem., 71:3894[1999]).

A 2-D gel provides a two dimensional separation from one initial loading of the cell lysate. The intensities of different spots on the same gel are representative of the relative protein abundances in the original lysate. However, in the IEF-NP RP HPLC methods of the present invention the proteins are loaded for the IEF and the HPLC separations so that the band intensities in the 2-D IEF-NP RP HPLC image depend on the amount of protein loaded to the HPLC from each Rotofor fraction. Since the amount of material in each Rotofor fraction is different, the total area of each chromatogram was scaled to represent the total amount of protein that was recovered for each Rotofor fraction (See, FIG. 3). The result is that the protein band intensities can be compared both within the Rotofor fraction and between the different fractions.

In some embodiments of the present invention, 2-D gel techniques are used side-by-side with IEF-NP RP HPLC. In embodiments where specific proteins are desired for further characterization, the gel can provide information indicating which fraction obtained with IEF-NP RP HPLC contains the desired protein or proteins.

IV. Isoelectric Focusing: Liquid vs. Gel Phase

The principal concern with liquid phase IEF is that the protein is not isoelectrically focused as effectively as it would be in a gel due to diffusion of the protein in solution. In the case of α-enolase, if one compares the liquid and gel phase images, it can be seen that in both cases substantial spreading of the protein occurs over a wide pI range. This range spans from pI 6.5 to pI 9.5 in both the liquid phase and the gel phase. For more acidic proteins such as β-actin, it appears that in the liquid phase the protein is more dispersed in the pI dimension than for the corresponding gel separated protein. Both methods provide a reasonably accurate assessment of the pI of the protein of interest. Referring to Table 1, it can be seen that as the Rotofor fraction pH increases, so generally does the pI of identified proteins therein. The pH of fraction 3 measures 4.2 and the proteins identified from this fraction range in pI from 4.09 to 5.7. The pH of fraction 9 was 5.8 and the proteins identified from that fraction ranged from 5.29 to 6.45. The pH of fraction 16 was 7.2 and the pI range of proteins found there ranged from 7.01 to 8.93. The pI accuracy therefore ranges from ±0.65 to 1.73 pI units. This is comparable to the carrier ampholyte based gel. It should be remembered that the pI of a given protein may vary significantly due to post-translational modifications such as phosphorylation and glycosylation, as well as to artifactual modifications such as carbamylation and oxidation.

V. Second Dimension Liquid Separation

Fraction 16, FIG. 4, may be used as an example of the quantification of isolated proteins. For fraction 16, the volume of injection was 160 μL. This means that if the concentration of protein was 201.4 μg/mL then the amount of protein loaded was 32.2 μg. The chromatogram was integrated using Microcal Origin software and the total area was determined to be 97.78. The areas of peaks 16E and 16J were 3.68 and 5.41 respectively. Dividing the peak area by the total area gives the fraction of protein represented by the peak. Therefore, if one assumes 100% protein recovery, the amount of PPIASE (16E, t_{R}=5.68) in 16 was (0.0376*32.2 μg) 1.21 μg and the amount of a-enolase (16J, t_{R}=7.45) was (0.0553*32.3 μg) 1.78 μg. The peak areas were generated by absorbance of 214 nm light at the amide bonds of the proteins and so should offer low selectivity thereby allowing for a good measure of the amount of protein in the peak regardless of the type of protein.

FIG. 4 shows how the continuous integration of the chromatogram may be used to estimate the amount of protein isolated in a given peak. The peak area line is simply converted into mass units from which the observer can measure the change in the vertical mass axis that occurs over the width of the peak of interest. If one knows the initial concentration of protein in the cell lysate and the number of cells that were lysed, a quantitative comparison of different cell lysates can be made. This comparison is important to studying changes in protein expression levels due to some disease state or pharmacological treatment. In gel work, a technique used for protein quantification in different samples is to normalize the integrated optical density of the spot of interest to that of standard proteins whose expression levels are thought to be constant. In this way any experimental variation in spot intensity can be corrected. This same method is applied to the IEF-NP RP HPLC image to allow for reliable quantification of proteins of interest such that changes in expression level are quantitatively observed.

The assumption in these experiments is 100% protein recovery. One can determine the actual % recovery of protein and the dependence on elution time. Typical protein recoveries have been shown to range from 70 to 95% in NP RP HPLC (Wall et al., Anal. Chem., 71:3894[1999]) and so, with a more likely percent recovery of 80%, the amount of PPIASE and α-enolase in fraction 16 would be estimated to be 1.0 μg and 1.42 μg, respectively.

VI. Rotofor Fraction Analysis by NP RP HPLC vs. 1-D SDS PAGE

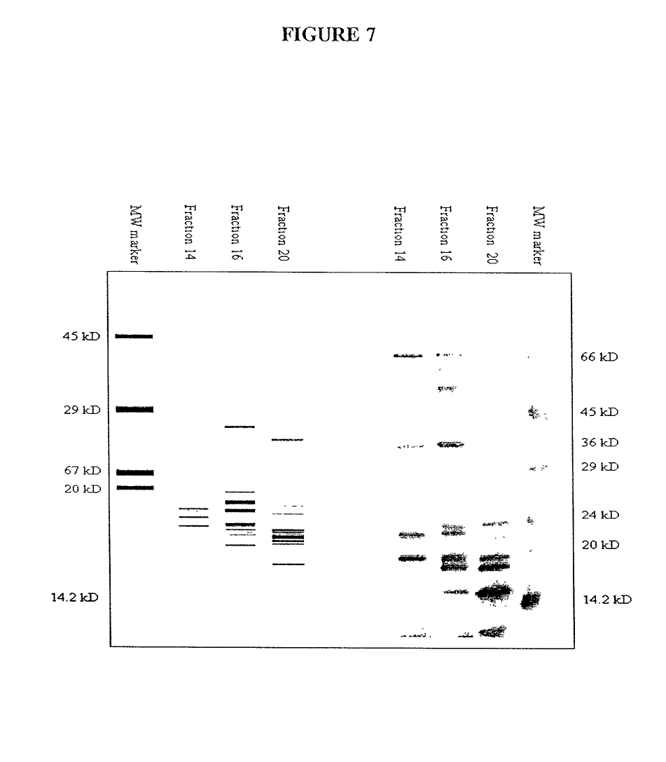

NP RP HPLC provides highly efficient protein separations (See e.g., Chen et al., Rap. Comm. Mass Spec., 12:1994[19981; Wall et al., Anal. Chem., 71:3894 [1999]; and Chong et al., Rap. Comm. Mass Spec., 13:1808[1999]), and is a far easier method to automate as compared to gels in terms of injection, data processing and protein collection. In addition the NP RP HPLC separations provided by the present invention are 70 times faster than the equivalent separation by 1-D SDS-PAGE, which requires 14 hours. In the experiments described above, the NP RP HPLC method has greater resolving power generating 35 bands where the 1-D gel generates only 26 bands. A direct comparison of the two methods, as shown in FIG. 7, reveals that the NP RP HPLC bands are much narrower than those of the 1-D SDS PAGE over a similar molecular weight range. Also it is clear that as molecular weight decreases, the 1-D gel band width increases substantially. In NP RP HPLC the opposite trend occurs where the lower molecular weight proteins show improved resolution and sensitivity. This image may appear to show that the NP RP HPLC separation fails with larger proteins as there are few bands in the upper region of the image. However, this is not the case as it is important to remember that the vertical dimension for NP RP HPLC is not protein molecular weight but rather protein hydrophobicity. This is evidenced by the observation of the elution of bovine serum albumin (66 kDa), a relatively hydrophilic protein, half way up an image.

VII. Elution Time Prediction for Known Target Protein

One of the advantages of the 2-D gel is that the vertical coordinate of the gel may be used to estimate the molecular weight of the protein with a ±10% error. The position of a protein of interest can therefore be estimated before the protein is identified from the gel. In an attempt to correlate elution time in the methods of the present invention with the mass of the protein, a linear fit to a plot of percent acetonitrile at time of elution (% B) versus the log(MWt)/protein polar ratio was generated. The polar ratio (PR) is the number of polar amino acids divided by the total number of amino acids in the protein and the molecular weight is in kDa. The proteins used for this plot were four of the standards listed in FIG. 1 as well as a sampling of six of the proteins from Table 1 (HSP60, β-actin, TIM, α-enolase, PPIASE and glyceraldehyde-3-phosphate). The resulting equation

%B/100=0.079805*(log MWt)/PR+0.077686, (R=0.9677, SD=0.014722, N=7)) (equation 1)

is used to predict the elution time of target proteins. For HSP60, β-actin and α-enolase the experimental elution times were 10.28, 10.15 and 7.25 respectively. The predicted elution times were 10.20, 10.13 and 9.78. In the cases of HSP60 and β-actin the prediction works well, whereas for x-enolase the prediction is not as good. While not precise, this prediction does give some idea of when a protein will elute such that a given target protein, for which the molecular weight and hydrophobicity are known, can be found more readily.

VIII. Protein Identification by Enzymatic Digestion, MALDI-TOF MS and MSFit Database Searching

The proteins that were identified from a representative sampling of the bands from the IEF-NP RP HPLC separation are listed in Table 1. A sampling of approximately 80 proteins from 12 of the Rotofor fractions were digested and their peptide mass maps successfully obtained by MALDI-TOF MS. Of these 80, 38 different proteins were identified. In this case, identifying roughly 50% of the proteins searched is to be expected as not all the proteins are in the available databases. Similar results were observed for proteins analyzed from 2-D gels of the HEL cell samples. The current table in Swiss-2DPAGE lists 19 protein entries for the HEL cell. Of these 19 proteins, five were identified from the IEF-NP RP HPLC separation. In the gel, these same five proteins were also identified.

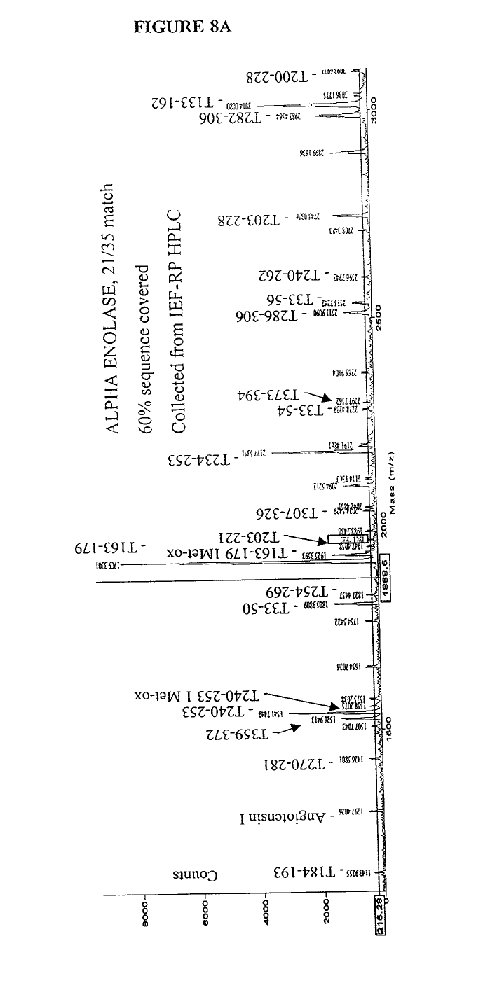

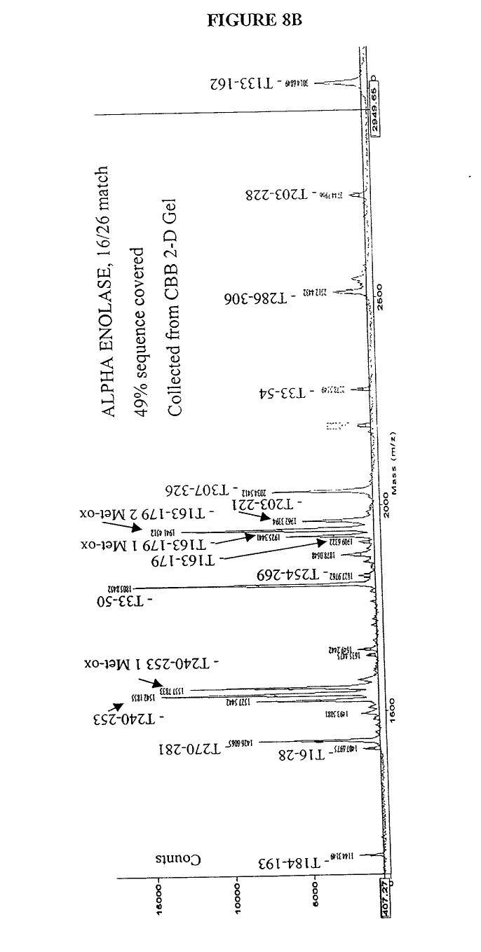

In general, it appears that the gel MSFit results are better than those from the liquid phase. This can be attributed to the fact that the gel proteins were reduced and alkylated with DTE and iodoacetamide respectively prior to the running of the second dimension. This step would help insure that all disulfide bonds are broken and optimal proteolysis is produced. Thus, this derivatization step can be added to the IEF-NP RP HPLC method, by performing the reduction and alkylation step prior to NP RP HPLC or during cell lysis. Nevertheless, in some cases the IEF-NP RP HPLC digestions surpassed those from the gel in coverage and quality. This is evidenced in FIG. 8, which shows a direct comparison of the MALDI-TOF MS for α-enolase as isolated via the IEF-NP RP HPLC method and the gel method. These mass spectra were calibrated externally at first and the mass profiles used to search the Swiss protein database with a mass accuracy of 400 ppm. These searches gave strong hits to o-enolase for both the gel and the liquid protein digests. Each mass spectrum was then recalibrated internally using matched peptide peaks from the initial externally calibrated match. The new peak table was then used to search the same Swiss protein database but with 200 ppm mass accuracy. FIG. 8 clearly shows that the digestion from the liquid phase is improved compared to that from the gel. The IEF-NP RP HPLC mass spectrum matches to 60% of the protein sequence whereas that from the gel matches to 49%. Achieving a match to 60% of the sequence of a 47 kDa protein is very unusual for MALDI-TOF MS analysis and represents a significant improvement over gel digests. Although the present invention is not limited to any particular mechanism, the increase in sequence coverage may be due to the fact that the protein is digested in the liquid phase, is relatively pure, and because the peptides are not lost due to being embedded inside the gel piece. Also if one observes the level of methionine oxidation in the peak that matches to T163-179, it is clear that the protein isolated by IEF-NP RP HPLC is far less oxidized than that from the gel.

Many of the NP RP HPLC chromatograms contain some peaks that are not fully resolved to baseline. This need not be a problem as partially resolved proteins can still be effectively identified using MALDI-TOF MS analysis. In Rotofor fraction 3 there are peaks at 10.15 minutes and 10.25 minutes (See, Table 1). These peaks are only resolved to 50% above the baseline and yet it is clear that the peak eluting at 10.15 minutes is β-actin and the peak eluting at 10.25 minutes is HSP-60. Note that the predicted elution times for these proteins are 10.13 and 10.20 minutes respectively. As proteins can be identified from partially resolved peaks, faster separations with more rapid gradients are possible. The reproducibility of the pattern of bands can be determined by looking at the retention times for particular proteins as observed from different Rotofor fractions. 8-actin elutes at 10.15 minutes in both fractions 3 and 9; α-enolase elutes at 7.25, 7.45 and 7.39 minutes in fractions 12, 16 and 20 respectively; and HSP-60 elutes at 10.28 and 10.25 minutes in fractions 3 and 4 respectively. Clearly, with ±0.1 minutes variation in the retention times, these separations are quite reproducible from run to run.

Thus, the methods of the present invention have been shown to provide advantageous methods for the reproducible separation of large numbers of proteins. In the human erythroleukemia cell lysate example, the methods are capable of resolving 700 bands with a rapid gradient, and 1000 bands with a longer gradient. There were 38 different proteins tentatively identified, by MALDI-TOF MS and MSFit database searching, after analysis of a fraction of these bands. This compares favorably with the 19 different proteins that have been identified to date from the 2-D gel. Some of the proteins found in the human erythroleukemia cell lysate; including α-enolase (Rasmussen et al., Electrophoresis 19:818[1998] and Mohammad et al., Enz. Prot., 48:37[1994]), glyceraldehyde-3-phosphate dehydrogenase (Bini et al., Electrophoresis 18:2832[1997] and Sirover, Biochim. Biophys. Acta 1432:159[1999]), NPM (Redner et al., Blood 87:882[1996]), CRKL (ten Hoeve et al., Oncogene 8:2469[1993]), and heat shock protein (HS27) (Fuqua et al., Cancer Research 49:4126[1989]), have been linked to various forms of cancer. NPM and CRKL have been linked specifically to leukemias.

The proteins identified in one exemplary experiment ranged from 12 kDa up to 75 kDa (although broader ranges are contemplated by the present invention); this range may include many of the proteins of interest to current research involving protein profiling, identification and correlation to some disease state or cell treatment. In sharp contrast to 2-D gels, this method is well-suited to automation. Mass spectrometric methods can be applied, such as ESI-MS and MALDI-TOF MS, to the detection of whole proteins and protein digests. Most importantly, the methods of the present invention provide an alternative 2-D protein map to the traditional 2-D gel and appears to improve results for lower mass proteins and more basic proteins. A key advantage of the liquid 2-D separation is that the end product is a purified protein in the liquid phase. Also, since the initial protein load can be fifty times that of the gel, the amount of a target protein that may be isolated by one IEF-NP RP HPLC separation is potentially fifty times higher than that obtainable from a 2-D gel separation. Additionally, in the case that the investigator is interested in specific proteins where the pI is known, this method may be used to isolate and identify the target protein in less than 24 hours, since only the fraction of interest need be analyzed via the second dimension separation. The gel-based method would require three days to achieve the same result.

IX. Identification of Novel Tumor Antigens

There is substantial interest in identifying tumor proteins that are immunogenic. Autoantibodies to tumor antigens and the antigens themselves represent two types of cancer markers that can be assayed in patient serum and other biological fluids. IEF-NP RP HPLC-MS has been implemented for the identification of tumor proteins that elicit a humoral response in patients with cancers. The identification of proteins that specifically react with sera from cancer patients was demonstrated using this approach. Solubilized proteins from a tumoral cell line are subjected to IEF-NP RP HPLC-MS. Individual fractions defined on the basis of pI range are subjected simultaneously to one-dimensional electrophoresis as well as to HPLC. Sera from cancer patients are reacted with Western blots of one-dimensional electrophoresis fractions. One band which reacted specifically with sera from lung cancer patients and not from controls was found to contain both Annexin II and aldoketoreductase. The ability to subfractionate further proteins contained in this fraction by HPLC led to the identification of Annexin II as the tumor antigen that elicited a humoral response in lung cancer patients.

X. Comparative Analysis

As is clear from the above description, the methods of the present invention offer the opportunity to compare protein profiles between two or more samples (e.g., cancer vs. control cells, undifferentiated vs. differentiated cells, treated vs. untreated cells). In one embodiment of the present invention, the two samples to be compared are run in parallel. The data generated from each of the samples is compared to determine differences in protein expression between the samples. The profile for any given cell type may be used as a standard for determining the identity of future unknown samples. Additionally, one or more proteins of interest in the expression pattern may be further characterized (e.g., to determine its identity). In an alternative embodiment, the proteins from the samples are run simultaneously. In these embodiments, the proteins from each sample are separately labeled so that, during the analysis stage, the protein expression patterns from each sample are distinguished and displayed. The use of selective labeling can also be used to analyze subsets of the total protein population, as desired.

As is clear from the above description, the methods and compositions of the present invention provide a range of novel features that provide improved methods for analyzing protein expression patterns. For example, the present invention provides methods that combine IEF, resulting in pI-focused proteins in liquid phase fractions, with nonporous RP HPLC to produce 2-dimensional liquid phase protein maps. The data generated from such methods may be displayed in novel and useful formats such as viewing a collection of different pI NP RP HPLC chromatograms in one 2-D image displaying the chromatograms in a top view protein band format, not the traditional side view peak format. As shown in FIG. 2, the side view peak format is shown to the left and the top view band format is shown to the right. The present invention also provides detergents that are compatible with automated systems employing multi-phase separation and detection steps.

The present invention provides additional characterization steps, including the identification of proteins separated by IEF-NP RP HPLC using enzymatic digestions and mass spectrometric analysis of the resulting peptide mass fingerprints. Proteins may be detected to determine their molecular weights by analyzing the effluent from the HPLC with either off-line collection to a MALDI plate (Perseptive) or on-line analysis using orthogonal extraction time-of-flight. The data generated from such methods may be displayed in novel and useful formats such as using the data from the MALDI or LCT generated protein molecular weights to generate total ion chromatograms (TIC) that would be virtually identical to the original UV-absorbance chromatograms. The signal of these chromatograms would be based on the number of ions generated from the HPLC effluent of a given group of pI-focused proteins, not by absorption of light. These chromatograms are plotted in the same 2-D top view band format as mentioned above. These methods allow one to fully integrate and deconvolute each of the TIC's generated to display complete mass spectra of each collection of pI-focused proteins. The methods also allow the display of all the 10 integrated TIC's in one 2-D image where the vertical dimension is in terms of protein molecular weight and the horizontal dimension is in terms of protein pI. The protein mass spectra appears as bands as they are also viewed from the top. This image would therefore also contain quantitative information (in the case of the LCT) and so the bands would vary in intensity depending on the amount of protein present.

The liquid phase methods for protein mass mapping would also allow for collection of protein fractions to microtubes such that the proteins could be digested and the peptide mass maps analyzed to determine the identity of said proteins simultaneously. Laser induced fluorescence (LIF) detection schemes are used in conjunction with this method to increase the overall sensitivity by three orders of magnitude. The liquid phase LIF detector provides more sensitive fluorescence detection than in the gel as there would be no gel background fluorescence. This LIF detection method could be used in a number of ways including, but not limited to:

1) Combining equal amounts of two cell lysates that have each been previously stained with a different fluorescent dye followed by use of a dual fluorescence detector to simultaneously detect the same proteins from two different cell lysates. This would allow for very accurate comparisons of the relative amounts of proteins found for different cell lines or tissues; and

2) Using a fluorescently tagged antibody to label specific target proteins in a cell lysate such that they can be targeted for thorough analysis without looking at all the other proteins.

The methods and apparatuses of the present invention also offer an efficient system for combining with other analysis techniques to obtain a thorough characterization of a given cell, tissue, or the like. For example, the methods of the present invention may be used in conjunction with genetic profiling technologies (e.g., gene chip or hybridization based nucleic acid diagnostics) to provide a fuller understanding of the genes present in a sample, the expression level of the genes, and the presence of protein (e.g., active protein) associated with the sample.

Experimental

The following example serves to illustrate certain preferred embodiments and aspects of the present invention and is not to be construed as limiting the scope thereof.

### EXAMPLE 1

HEL Cell Sample Preparation

The human erythroleukemia (HEL) cell line was obtained from the Department of Pediatrics at The University of Michigan. HEL cells were cultured (7% CO₂, 37° C.) in RPMI-1640 medium (Gibco) containing 4 mM glutamine, 2 mM pyruvate, 10% fetal bovine serum (Gibco), penicillin (100 units per mL), streptomycin (100 units per mL) and 250 mg of hygromycin (Sigma). The HEL cell pellets were washed in sterile PBS, and then stored at −80° C. The cell pellets were then re-suspended in 0.1% n-octyl β-D-galactopyranoside (OG) (Sigma) and 8 M urea (Sigma) and vortexed for 2 minutes to effect cell disruption and protein solubilization. The whole cell protein extract was then diluted to 55 mL with the Rotofor buffer and introduced into the Rotofor separation chamber (Biorad).

### EXAMPLE 2

1-D Gel and SDS PAGE Separation

HEL cell proteins, resolved by Rotofor separation into discrete pI ranges, were further resolved according to their apparent molecular weight by SDS-PAGE. This procedure takes approximately 14 hours to complete. Samples of rotofor fractions were suspended in an equal volume of sample buffer (125 mM Tris (pH 6.8) containing 1% SDS, 10% glycerol, 1% dithiothreitol and bromophenol blue) and boiled for 5 min. They were then loaded onto 10% acrylamide gels. The samples were electrophoresed at 40 volts until the dye front reached the opposite end of the gel. The resolved proteins were visualized by silver staining. The gels were fixed overnight in 50% ethanol containing 5% glacial acetic acid, then washed successively (for 2 hours each) in 25% ethanol containing 5% glacial acetic acid, 5% glacial acetic acid, and 1% glacial acetic acid. The gels were impregnated with 0.2% silver nitrate for 25 min. and were developed in 3% sodium carbonate containing 0.4% formaldehyde for 10 min. Color development was terminated by impregnating the gels with 1% glacial acetic acid, after which the gels were digitized.

### EXAMPLE 3

2-D PAGE

In order to prepare protein extracts from the HEL cells, the harvested cell pellets were lysed by addition of three volumes of solubilization buffer consisting of 8 M urea, 2% NP-40, 2% carrier ampholytes (pH 3.5 to 10), 2% β-mercaptoethanol and 10 mM PMSF, after which the buffer containing the cell extracts was transferred into microcentrifuge tubes and stored at −80° C. until use.

Extracts of the cultured HEL cells were separated in two dimensions as previously described by Chen et al. (Chen et al., Rap. Comm. Mass Spec. 13:1907 [1999]) with some modifications as described below. Subsequent to cellular lysis in solubilization buffer, the cell lysates from approximately 2.5×10⁶ cells were applied to isoelectric focusing gels. Isoelectric focusing was conducted using pH 3.5 to 10 carrier ampholytes (Biorad) at 700 V for 16 h, followed by 1000 V for an additional 2 hours. The first dimension tube gel was soaked in a solution of 2 mg/mL of dithioerythritol (DTE) for 10 minutes, and then soaked in a solution of 20 mg/mL of iodoacetamide (Sigma) for 10 minutes, both at room temperature. The first-dimension tube gel was loaded onto a cassette containing the second dimension gel, after equilibration in second-dimension sample buffer (125 mM Tris (pH 6.8), containing 10% glycerol, 2% SDS, 1% dithioerythritol and bromophenol blue). For the second-dimension separation, an acrylamide gradient of 11.5% to 14% was used, and the samples were electrophoresed until the dye front reached the opposite end of the gel. The separated proteins were transferred to an Immobilon-P PVDF membrane. Protein patterns in some gels were visualized by silver staining or by Coomassie blue staining, and on Immobilon-P membranes by Coomassie blue staining of the membranes.

### EXAMPLE 4

Rotofor Isoelectric Focusing

A preparative scale Rotofor (Biorad) was used in the first dimension separation. This device separated the proteins in liquid phase according to their pI, and is capable of being loaded with up to a gram of protein, with the total buffer volume being 55 mL. Alternatively, for analysis of smaller quantities of protein, a mini-Rotofor with a reduced volume can be used. These proteins were separated by isoelectric focusing over a 5 hour period where the separation temperature was 10° C. and the separation buffer contained 0.1% n-octyl β-D-galactopyranoside (OG) (Sigma), 8 M urea (ICN), 2% β-mercaptoethanol (Biorad) and 2.5% Biolyte ampholytes, pH 3.5-10 (Biorad). The procedure used for running the Rotofor (Rotofor Purification System, Biorad) was of the standard procedure described in the manual from Biorad as modified herein. The 20 fractions contained in the Rotofor were collected simultaneously, into separate vials using a vacuum source attached by plastic tubing to an array of 20 needles, which were punched through a septum. The Rotofor fractions were aliquotted into 400 μL amounts in polypropylene microcentrifuge tubes and could be stored at −80° C. for further analysis if necessary. An advantage of gel methods is the ability to store proteins stably in gels at 4° C. for further use. The concentration of protein in each fraction was determined via the Biorad Bradford based protein assay. The pH of the fractions was determined using pH indicator paper (Type CF, Whatman).

### EXAMPLE 5

NP RP HPLC

Separations were performed at a flow rate of 1.0 mL/minute on an analytical (4.6*14 mm) NP RP HPLC column containing 1.5 μm C18 (ODSI) non-porous silica beads (Micra Scientific Inc.). The column was placed in a Timberline column heater and maintained at 65° C. The separations were performed using water/acetonitrile (0.1% TFA, 0.05% OG) gradients. The gradient profile used was as follows: 1) 0 to 25% acetonitrile (solvent B) in 2 minutes; 2) 25 to 35% B in 2 minutes; 3) 35 to 45% B in 5 minutes; 4) 45 to 65% B in 1 minute; 5) 65 to 100% B in 1 minute; 6) 100% B in 3 minutes; 7) 100 to 5% B in 1 minute. The start point of this profile was one minute into the gradient due to a one-minute dwell time. The acetonitrile was 99.93+% HPLC grade (Sigma) and the TFA were from 1 mL sealed glass ampules (Sigma). The non-ionic detergent used was n-octyl β-D-galactopyranoside (OG) (Sigma). The HPLC instrument used was a Beckman model 127s/166. Peaks were detected by absorbance of radiation at 214 nmn in a 15 μL analytical flow cell.

Protein standards (Sigma) used as MW protein markers and for correlation of retention time, molecular weight and hydrophobicity were bovine serum albumin (66 kDa), carbonic anhydrase (29 kDa), ovalbumin (45 kDa), lysozyme (14.4 kDa), trypsin inhibitor (20 kDa) and cc-lactalbumin (14.2 kDa).

### EXAMPLE 6

MALDI-TOF MS of NP RP HPLC Isolated Proteins

The MALDI-TOF MS analyses were performed on a Perseptive Voyager Biospectrometry Workstation equipped with delayed extraction technology, a one-meter flight tube and a high current detector. The N₂ laser provided light at 337 nm for laser desorption and ionization. MALDI-TOF MS was used to determine masses of peptides from protein digests using a modified (described herein) version of the two layer dried droplet method of Dai et al. (Dai et al., Anal. Chem., 71:1087[1999]). The MALDI matrix α-cyano-4-hydroxy-cinnamic acid (α-CHCA) (Sigma Chemical Corp., St Louis, Mo., USA) was prepared in a saturated solution of acetone (1% TFA). This solution was diluted 8-fold in the same acetone solution (1% TFA) and then added to the sample droplet in a 1:2 ratio (v:v). The mixed droplet was then allowed to air dry on the MALDI plate prior to introduction into the MALDI TOF instrument for molecular weight analyses.

The proteins were collected into 1.5 mL polypropylene micro-tubes containing 20 μL of 0.8% OG in 50% ethanol. In preparation for enzymatic digestion the acetonitrile was removed via speedvac at 45° C. for 30 minutes. A solution of 200 mM NH₄HCO₃ (ICN)/1 mM β-mercaptoethanol was then added in a 1 to 2 ratio to the remaining solution in the tubes, resulting in a solution of 50 to 100 mM NH₄HCO₃ with a total volume of approximately 150 μL. Subsequently 0.25 μg of enzyme was added to this solution and then the mixture was vortexed and placed in a 37° C. warm room for 24 hours. The enzymes used were either trypsin (Promega, TPCK treated), which cleaves at the carboxy side of the arginine and lysine residues, or Glu-C (Promega), which in 50-100 mM NH₄HCO₃ solution cleaves at the carboxy side of the glutamic acid residues.

The digest solutions were typically 100 μL in volume and 30 to 50 μL of this solution was desalted and concentrated to a final volume of 5 μL using Zip-Tips (Millipore) with 2 μL C18 resin beds. The purified peptide solution was then used to spot onto the MALDI plate for subsequent MALDI-TOF MS analysis. All spectra were obtained with 128 averages and internally or externally calibrated using the PerSeptive standard peptide mixture containing angiotensin I, ACTH(1-17), ACTH(18-39) and ACTH(7-38) (PerSeptive Biosystems).

These digests were then used to aid in the identification of the proteins by MALDI-TOF MS analysis and MSFit database searching (Wall et al., Anal. Chem., 71:3894[19991). The peptide mass maps were searched against the Swiss and NCBInr protein databases using MSFit allowing for 2 missed cleavages. The molecular weight ranged from 5 kDa to 70 kDa and the pI ranged over the full pI range. Externally calibrated peptide masses were searched with 400 ppm mass accuracy and internally calibrated peptide masses were searched with 200 ppm mass accuracy.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

We claim:

1. A method for separating proteins comprising:
a) providing:
i) a sample comprising a plurality of proteins;
ii) a first separating apparatus that separates proteins based on charge; and
iii) a second separating apparatus comprising non-porous reverse phase HPLC;
b) treating said sample with said first separating apparatus to produce a first separated protein sample; and
c) treating at least a portion of said first separated protein sample with said second separating apparatus to produce a second separated protein sample.

2. The method of claim 1, further comprising the step of d) displaying at least a first physical property of at least a portion of said second separated protein sample.

3. The method of claim 2, wherein said displaying comprises a schematic representation of first and second physical properties of at least a portion of said plurality of proteins.

4. The method of claim 3, wherein said first and second properties comprise pI and hydrophobicity.

5. The method of claim 4, wherein said displaying further comprises representing a third physical property of at least a portion of said plurality of proteins.

6. The method of claim 5, wherein said third physical property comprises protein mass.

7. The method of claim 1, wherein said sample comprising a plurality of proteins further comprises a buffer, wherein said plurality of proteins are solubilized in said buffer and wherein said buffer is compatible with said first and said second separating apparatus.

8. The method of claim 7, wherein said buffer is further compatible with mass spectrometry.

9. The method of claim 7, wherein said buffer comprises a compound of the formula n-octyl C₆-C₁₂ glycopyranoside.

10. The method of claim 9, wherein said compound of the formula n-octyl C₆-C₁₂ glycopyranoside is selected from n-octyl β-D-glucopyranoside and n-octyl β-D-galactopyranoside.

11. The method of claim 1, wherein said sample comprises a cell lysate.

12. The method of claim 1, wherein said first separating apparatus comprises a liquid phase separating apparatus.

13. The method of claim 1, wherein said first separating apparatus comprises an isoelectric focusing apparatus.

14. The method of claim 1, wherein said second separating apparatus comprises non-porous C18 silica beads.

15. The method of claim 1, further comprising the step of d) determining the identity of at least one protein of said second separated protein sample.

16. The method of claim 15, wherein said determining the identity of at least one protein comprises analyzing said at least one protein from said second separated protein sample with mass spectrometry.

17. The method of claim 1, wherein said treating said sample with said first separating apparatus to produce a first separated protein sample comprises loading at least 25 mg of protein into said first separating apparatus.

18. A method for separating proteins comprising:
a) providing:
i) a first separating apparatus that separates proteins based on a first physical property;
ii) a second separating apparatus that separates proteins based on a second physical property;
iii) a mass spectroscopy apparatus; and
iv) a sample comprising a plurality of proteins, said sample comprising a buffer, wherein said plurality of proteins are solubilized in said buffer and wherein said buffer is compatible with said first separating apparatus, said second separating apparatus, and said mass spectroscopy apparatus;
b) treating said sample with said first separating apparatus to produce a first separated protein sample;
c) treating at least a portion of said first separated protein sample with said second separating apparatus to produce a second separated protein sample; and
d) mass spectrally analyzing at least a portion of said second separated protein sample with said mass spectroscopy apparatus to characterize masses of proteins in said second separated protein sample.

19. The method of claim 18, further comprising the step of e) displaying at least a first property of at least a portion of said second separated protein sample.

20. The method of claim 18, wherein said buffer comprises a compound of the formula n-octyl C₆-C₁₂ glycopyranoside.

21. The method of claim 20, wherein said compound of the formula n-octyl C₆-C₁₂ glycopyranoside is selected from n-octyl β-D-glucopyranoside and n-octyl β-D-galactopyranoside.

22. The method of claim 18, wherein said sample comprises a cell lysate.

23. The method of claim 18, wherein said first separating apparatus comprises a liquid phase separating apparatus.

24. The method of claim 23, wherein said first separating apparatus comprises an isoelectric focusing apparatus.

25. The method of claim 18, wherein said second separating apparatus comprises reverse phase HPLC.

26. The method of claim 25, wherein said reverse phase HPLC comprises non-porous reverse phase HPLC.

27. The method of claim 26, wherein said reverse phase HPLC comprises non-porous reverse phase HPLC comprises non-porous C18 silica beads.

28. The method of claim 18, further comprising the step of e) determining the identity of at least one protein of said second separated protein sample.

29. The method of claim 18, wherein said first and said second physical properties are selected from charge, hydrophobicity, or molecular weight.

30. The method of claim 19, wherein said displaying comprises providing a representation of first and second physical properties of at least a portion of said plurality of proteins.

31. The method of claim 30, wherein said first and second properties comprise pI and hydrophobicity.

32. The method of claim 30, wherein said displaying further comprises representing a third physical property of at least a portion of said plurality of proteins.

33. The method of claim 32, wherein said third physical property comprises protein mass.

34. The method of claim 18, wherein said treating said sample with said first separating apparatus to produce a first separated protein sample comprises loading at least 25 mg of protein into said first separating apparatus.

35. A method for displaying separated proteins comprising:
a) providing:
i) a first separating apparatus that separates proteins based on a first physical property;
ii) a second separating apparatus that separates proteins based on a second physical property;
iii) a mass spectroscopy apparatus; and
iv) a sample comprising a plurality of proteins;
b) treating said sample with said first separating apparatus to produce a first separated protein sample;
c) treating at least a portion of said first separated protein sample with said second separating apparatus to produce a second separated protein sample;
d) mass spectrally analyzing at least a portion of said second separated protein sample with said mass spectroscopy apparatus; and
e) displaying at least a portion of said second separated protein sample, wherein said displaying provides a representation of said first physical property, said second physical property, and relative protein abundance of at least a portion of said plurality of proteins.

36. The method of claim 35, wherein said first and second properties comprise pI and hydrophobicity.

37. The method of claim 35, wherein said sample comprising a plurality of proteins further comprises a buffer, wherein said plurality of proteins are solubilized in said buffer and wherein said buffer is compatible with said first and said second separating apparatus.

38. The method of claim 37, wherein said buffer is further compatible with mass spectrometry.

39. The method of claim 38, wherein said buffer comprises a compound of the formula n-octyl C₆-C₁₂ glycopyranoside.

40. The method of claim 39, wherein said compound of the formula n-octyl C₆-C₁₂ glycopyranoside is selected from n-octyl β-D-glucopyranoside and n-octyl β-D-galactopyranoside.

41. The method of claim 35, wherein said sample comprises a cell lysate.

42. The method of claim 35, wherein said first separating apparatus comprises a liquid phase separating apparatus.

43. The method of claim 42, wherein said first liquid phase separating apparatus comprises an isoelectric focusing apparatus.

44. The method of claim 35, wherein said second separating apparatus comprises reverse phase HPLC.

45. The method of claim 44, wherein said reverse phase HPLC comprises non-porous reverse phase HPLC.

46. The method of claim 45, wherein said reverse phase HPLC comprises non-porous reverse phase HPLC comprises non-porous C18 silica beads.

47. The method of claim 35, further comprising the step of f) determining the identity of at least one protein of said second separated protein sample.

48. The method of claim 35, wherein said first and said second physical properties are selected from charge, hydrophobicity, or molecular weight.

49. The method of claim 35, wherein said treating said sample with said first separating apparatus to produce a first separated protein sample comprises loading at least 25 mg of protein into said first separating apparatus.

50. A method for comparing protein expression patterns comprising:
a) providing:
i) first and second samples comprising a plurality of proteins;
ii) a first separating apparatus that separates proteins based on charge; and
iii) a second separating apparatus comprising non-porous reverse phase HPLC;
b) treating said first and second samples with said first separating apparatus to produce first and second separated protein samples;
c) treating at least a portion of said first and second separated protein samples with said second separating apparatus to produce third and fourth separated protein samples;
d) displaying at least a portion of said third and said fourth separated protein samples under conditions such that first and second physical properties of said third and fourth separated proteins samples are represented; and
e) comparing said first and second physical properties of said third separated protein sample with said first and second physical properties of said fourth separated protein sample.

51. The method of claim 50, wherein said first and said second samples comprising a plurality of proteins further comprise a buffer, wherein said plurality of proteins are solubilized in said buffer and wherein said buffer is compatible with said first and said second separating apparatus.

52. The method of claim 51, wherein said buffer is further compatible with mass spectrometry.

53. The method of claim 52, wherein said buffer comprises a compound of the formula n-octyl C₆-C₁₂ glycopyranoside.

54. The method of claim 53, wherein said compound of the formula n-octyl C₆-C₁₂ glycopyranoside is selected from n-octyl β-D-glucopyranoside and n-octyl β-D-galactopyranoside.

55. The method of claim 50, wherein said first and said second samples are combined into a single sample prior to step b).

56. The method of claim 50, wherein at least a portion of said proteins in said first sample comprise a first label and wherein at least a portion of said proteins in said second sample comprises a second label.

57. The method of claim 50, wherein said first and said second samples comprise cell lysates.

58. The method of claim 50, wherein said first separating apparatus comprises a liquid phase separating apparatus.

59. The method of claim 58, wherein said liquid phase first separating apparatus comprises an isoelectric focusing apparatus.

60. The method of claim 50, wherein said second separating apparatus comprises non-porous C18 silica beads.

61. The method of claim 50, further comprising the step of f) determining the identity of at least one protein of said third or said fourth separated protein samples.

62. The method of claim 61, wherein said determining the identity of at least one protein comprises analyzing said at least one protein from said third or said fourth separated protein sample with mass spectrometry.

63. The method of claim 1, wherein said treating said sample with said first separating apparatus to produce a first separated protein sample comprises loading at least 25 mg of protein into said first separating apparatus.

64. A protein separating system comprising:
a) a first separating apparatus that separates proteins based on charge;
b) a first delivery apparatus configured to receive separated protein from said first separating apparatus; and
c) a second separating apparatus comprising non-porous reverse phase HPLC, wherein said second separating apparatus is configured to receive proteins from said first delivery apparatus.

65. The system of claim 64, further comprising d) a detection system that detects proteins separated by said second separating apparatus.

66. The system of claim 65, further comprising e) a processor connected to said detection system, wherein said processor produces a data representation of detected proteins.

67. The system of claim 65, further comprising f) a display system that displays said data representation, wherein first and second physical properties of at least a portion of said proteins produced by said second separating apparatus are represented.

68. The system of claim 64, wherein said first separating apparatus comprises a liquid phase separating apparatus.

69. The system of claim 68, wherein said liquid phase first separating apparatus comprises an isoelectric focusing apparatus.

70. The system of claim 64, wherein said second separating apparatus comprises non-porous C18 silica beads.

71. The system of claim 64 further comprising:
d) a second delivery apparatus configured to receive separated protein from said second separating apparatus; and
e) a mass spectrometry apparatus configured to receive protein from said second delivery apparatus.

72. A system for displaying separated proteins comprising:
a) a first separating apparatus that separates proteins based on a first physical property;
b) a first delivery apparatus configured to receive separated protein from said first separating apparatus;
c) a liquid phase second separating apparatus that separates proteins based on a second physical property, and wherein said second separating apparatus is configured to receive proteins from said first delivery apparatus;
d) a detection system that detects proteins separated by said second separating apparatus;
e) a processor configured to run protein display software, wherein said protein display software produces a data representation of detected proteins; and
f) a display system that displays said data representation, wherein said first physical property, said second physical properties, and protein abundance of at least a portion of said plurality of proteins are represented.

73. The system of claim 72, wherein said processor is further configured to access a database, wherein said software is further configured to compare at least a portion of said data representation to protein information contained in said database.

74. The system of claim 72, wherein said first separating apparatus comprises a liquid phase separating apparatus.

75. The system of claim 74, wherein said liquid phase first separating apparatus comprises an isoelectric focusing apparatus.

76. The system of claim 72, wherein said second separating apparatus comprises a reverse phase HPLC apparatus.

77. The system of claim 74, wherein said reverse phase HPLC apparatus comprises a non-porous reverse phase HPLC apparatus.

78. The system of claim 77, wherein said non-porous reverse phase HPLC apparatus comprises non-porous C18 silica beads.

79. The system of claim 74, further comprising:
g) a second delivery apparatus configured to receive separated protein from said second separating apparatus; and
h) a mass spectrometry apparatus configured to receive protein from said second delivery apparatus.
